(19) United States
(12) Patent Application Publication
(10) Pub. No.: US 2011/0190332 A1
(43) Pub. Date: Aug. 4, 2011

(54) DOPAMINE RECEPTOR LIGANDS WITH ENHANCED DURATION OF ACTION

(75) Inventor: Richard B. Mailman, Hershey, PA (US)

(73) Assignee: EFFIPHARMA, Chapel Hill, NC (US)

(21) Appl. No.: 13/056,651

(22) PCT Filed: Jul. 31, 2009
(86) PCT No.: PCT/US2009/052334

§ 371 Date: Apr. 7, 2011

Related U.S. Application Data
(60) Provisional application No. 61/086,398, filed on Aug. 5, 2008.

(51) Int. Cl. A61K 31/473; C07D 221/18; A61P 25/16; A61P 25/18; A61P 25/28; A61P 25/22; A61P 25/00; A61P 25/30; A61P 25/14

(52) U.S. Cl. 514/284; 546/61

(57) ABSTRACT

Trans-hexahydrobenzoaphenanthridines of the formula (I) wherein X, Y, R₁, R₂, R₃, R₄, R₅, R₆, and R₇ are as defined herein, are disclosed. Pharmaceutical formulations including such compounds, and methods of using such compounds for treating a patient suffering from dopamine-related dysfunction of the central or peripheral nervous system are also disclosed.

This application claims priority to U.S. Provisional Application 61/086,398, the contents of which are hereby incorporated by reference for all purposes.

This invention was made with Government support under Grant No. MH-40537 awarded by the National Institute of Health. The Government has relinquished rights in the invention.

### FIELD OF THE INVENTION

The present invention relates to novel ligands for dopamine receptors, in particular, the dopamine D₁ receptor. More particularly, this invention is directed to certain substituted transhexahydrobenzo[a]phenanthridine and related compounds useful as selective D₁ dopamine receptor agonists, with relatively long duration of action and a relatively low likelihood of developing tolerance. The compounds can be used to treat dopamine-related dysfunction of the central nervous system and selected peripheral systems.

### BACKGROUND OF THE INVENTION

Dopamine receptors are divided into two pharmacological families labeled as D₁ and D₂ (Garau et al., 1978; Kebabian and Calne, 1979) that are now known to be coded by five genes, the products of which are all members of what is termed either the 7 transmembrane (7TM) or G protein-coupled receptor (GPCR) superfamily. The D₂-like family is coded by three different genes yielding D₂, D₃ and D₄ receptors. The other two genes code for the D₁ and D₅ (Dearry et al., 1990; Monsma et al., 1990; Sunahara et al., 1990; Zhou et al., 1990) (Sunahara et al., 1991; Tiberi et al., 1991), the ones of immediate importance to this application. The dopamine receptors have been the subject of numerous reviews and books (Huang et al., 2001; Jenner and Demirdemar, 1997; Neve and Neve, 1997; Neve et al., 2004; Sealfon and Olanow, 2000). For the purposes herein, references to D₁ or D₂ agonist shall refer to actions at the pharmacological subclasses (e.g., D₁ means the D₁-like receptors D₁ and D₅; and D₂ means the D₂-like D₂, D₃, and D₄) unless otherwise specified.

First described in the early 19th century (Parkinson, 1817), PD is the disorder with the clearest link to dopamine dysfunction. The motor effects are due principally to degeneration of the dopaminergic cells in the substantia nigra pars compacta, with consequent loss of dopamine terminals in the striatum. Numerous etiological mechanisms have been hypothesized to be involved, including environmental chemicals, genes, viral or other infectious agents, and the interaction of these factors. Whereas parkinsonism clearly can be induced by such insults (e.g., from chemicals like MPTP), most of PD is termed idiopathic or sporadic PD because there is no proven single etiology. With the discovery of the existence and function of dopamine (Carlsson, 1959; Carlsson et al., 1958), post-mortem studies revealed that PD was a dopamine deficiency disease (Ehringer and Hornykiewicz, 1960; Hornykiewicz, 1963). Although dopamine does not cross the blood-brain barrier, its precursor L-dopa (levodopa) was found to provide extraordinary responses at high doses (Cotzias et al., 1969), and became (and has remained) the “gold standard” of symptomatic benefit (Mailman and Huang, 2007). Levodopa is dramatically effective for several years early in the clinical disease, and its efficacy can be improved with adjunctive therapies including decarboxylase inhibitors (carbidopa and benserazide), COMT inhibitors (entacapone and tolcapone), and MAO-B inhibitors (selegiline and rasagiline). Parkinson's patients invariably take drugs that are such combination products, and well as additional adjunctive agents. Yet despite the utility of these drug combinations, the dopamine replacement approach clearly begins to fail within a decade or less, with concomitant decreases in efficacy and increases in side effects (e.g., on-off phenomena; dyskinesias). One approach that has been considered is to bypass the need for the metabolic conversion of levodopa to dopamine by using direct-acting dopamine agonists.

Despite the fact that high levels of D₁ binding are found in the brain in areas affected by Parkinson's disease (e.g., the caudate-putamen, entopeduncular and subthalamic nuclei, substantia nigra pars reticulata), for many years it was assumed that the D₂-like receptors were of much greater pharmacological importance in Parkinson's disease (Cederbaum and Schleifer, 1990). In 1991, dihydrexidine, the first high affinity full D₁ agonist, was used to provide compelling evidence that activation of D₁ receptors played a major role in alleviating the symptoms of Parkinson's disease (Huang et al., 2001; Mailman and Nichols, 1998; Taylor et al., 1991). In contrast, it has also been shown that, at least with respect to Parkinson's disease, that selective dopamine D₂ agonists have a much lesser effect than D₁ agonists. Other researchers have since demonstrated the utility of full D₁ agonists in the treatment of parkinsonism caused by MPTP in monkeys, and in two human Parkinson's clinical studies (Rascol et al., 1999; Rascol et al., 2001). To date, D₁ full agonists are the only drugs with efficacy comparable to that of levodopa in either the MPTP-monkey model or in humans. Unfortunately, the characteristics of dihydrexidine (too short acting) and other D₁ full agonists (tolerance and/or seizures) have precluded them from reaching the clinic (Mailman et al., 2001).

Currently there are several drugs approved for human use for Parkinson's disease. In the United States, these include pramipexole, ropinirole, rotigitine, and apomorphine. All of these compounds have highest affinity for D₂ dopamine receptors, and with the exception of apomorphine, they all have relatively modest efficacy in Parkinson's disease when used alone. On the other hand, apomorphine is very effective as monotherapy, and also is the only approved antiparkinson drug that has high D₁ intrinsic activity. Unfortunately, apomorphine, like dihydrexidine, has a very short duration of action. In addition, its D₂ properties decrease its tolerability (e.g., nausea and emesis). There thus remains a pressing need for a full D₁ agonist for symptomatic treatment until a true cure or preventative could be found for PD.

Dopamine also has been implicated in numerous other neurological and psychiatric disorders. For example, it has been hypothesized that excess stimulation of dopamine receptor subtypes may be linked to positive symptoms of schizophrenia, and dopamine deficiency may be related to negative symptoms or cognitive deficits. Additionally, it is generally recognized that alterations in dopaminergic function in the central nervous system and peripheripy may affect the signs and symptoms of attention deficit hyperactivity disorder (ADHD), Alzheimer's disease, autism, other types of cognitive impairment, hypertension, narcolepsy, substance abuse, and other behavioral, neurological, psychiatric, and physiological disorders.

CNS disorders include both psychiatric and neurological disorders. CNS disorders can be caused or influenced by genetic factors, chemical or drug exposure, infection, trauma, and other environmental factors or be of unknown etiology. CNS disorders include psychiatric and neurological diseases; and include neurodegenerative diseases, behavioral disorders, cognitive disorders, and affective disorders. Sometimes their clinical manifestations may result from inappropriate levels of neurotransmitter release, inappropriate properties of neurotransmitter receptors, and/or inappropriate interaction between neurotransmitters and their receptors. Several CNS disorders involve alterations in dopamine function, or can be treated symptomatically or prophylactically with drugs that affect dopamine function. Such CNS disorders include Parkinson's disease, schizophrenia, tardive dyskinesia, attention deficit hyperactivity disorder, substance abuse, autism, disorders with cognitive dysfunction (such as aging or Alzheimer's disease), Huntington's chorea, anxiety, mood disorders, and Tourette's syndrome among others.

It would be desirable to provide new compounds and methods for treating and preventing conditions such as CNS disorders by administering a dopamine agonist or a dopamine drug that is functionally selective (Urban et al., 2007) to a patient susceptible to, or suffering from, such a condition or disorder. It would be highly beneficial to provide individuals suffering from certain disorders (e.g., CNS diseases) with interruption of the symptoms of those disorders by administering a pharmaceutical composition containing an active ingredient having dopamine pharmacology, and which has a beneficial effect (e.g., upon the functioning of the CNS) but which does not provide any significant associated side-effects. It would be highly desirable to provide a pharmaceutical composition incorporating a compound which interacts with dopamine receptors, such as those which have the potential to affect the functioning of the CNS, but which compound when employed in an amount sufficient to affect the functioning of the CNS, does not significantly affect those receptor subtypes which have the potential to induce undesirable side effects (e.g., emesis and nausea or psychotic-like effects).

The present invention provides such compounds and methods of treatment and prevention.

### SUMMARY OF THE INVENTION

Novel C₂, C₃, and/or C₄-substituted trans-5,6,6a,7,8,12b-hexahydrobenzo[a]phenanthridines, oxa-, thio, and azo substituted analogs thereof, compositions including these compounds, and methods of treatment using the compounds, are disclosed. In one embodiment, the compounds include common structural features with those compounds described in U.S. Pat. Nos. 5,047,536 and 5,420,134, the contents of which are hereby incorporated by reference. They are particularly related to the compound trans-10,11-dihydroxy-5,6,6a,7,8,12b-hexahydrobenzo[a]phenanthridine (dihydrexidine), a D₁ agonist which has been the subject of much study over the past several years, except that the compounds described herein have an enhanced duration of action relative to the compounds in the '536 and '134 patents.

In one embodiment, the compounds described herein are 2-substituted analogs of the dihydrexidine series. The compounds in the '536 and '134 patents are, in some cases, full D₁ dopamine agonists, and in other cases, analogs with varying degrees of D₁:D₂ binding affinity, with the ability to differentially activate functions mediated by the dopamine D₂ receptor. All of the tested reported examples, however, were limited as potential drugs by extremely short durations of action. By adding small substituents to the 2-position, the compounds described herein have marked and unexpected increases in duration of action without markedly altering the receptor profile of the parent compound. While not wishing to be bound to a particular theory, it is believed that this is due to intrinsic steric protection against the conjugating activity of enzymes directed at the catechol moiety, and is analogous to adding a metabolism inhibitor to the primary drug.

The biological activities of the compounds described herein are known to vary significantly in their selectivity for the dopamine receptor subtypes, depending on the nature and positioning of the substituent groups. Substitution at the C₂, C₃, and/or C₄ position on the benzophenanthridine ring system provides a means for controlling receptor affinity and concomitantly receptor selectivity (Knoerzer et al., 1995). In addition, dihydrexidine has been shown to have unusual functional properties at the D₂ dopamine receptor called “functional selectivity” (Kilts et al., 2002; Mailman, 2007; Mottola et al., 2002; Urban et al., 2007). The ability to utilize these unique properties has been hindered by the extremely short duration of action of these compounds, a limitation overcome by this invention.

The present compounds can be administered, for example, by oral or parenteral routes of administration in amounts effective to evoke therapeutic responses in patients suffering from a variety of disorders, for example, central nervous disorders associated with dopamine release. Representative disorders include Parkinson's disease, cognitive impairment including that occurring in Alzheimer's disease, attention deficit disorder, narcolepsy, schizophrenia, autism, substance abuse, other centrally-mediated psychiatric and neurologic disorders, and hypertension and pulmonary function. In addition, the compounds can be used to improve the cognitive function of “normal” patients (i.e., those without frank clinical manifestations of cognitive deficit).

One representative compound is trans-2-methyl-5,6,6a, 7,8,12b-hexahydrobenzo[a]phenanthridine, the 2-methyl analog of dihydrexidine herein referred to as Compound 1.

Additional objects, and advantages of the invention, will become apparent to those skilled in the art upon consideration of the following detailed description of preferred embodiments exemplifying the best mode of carrying out the invention.

### BRIEF DESCRIPTION OF THE DRAWINGS

FIG. 1 is a graph showing the duration of activity of Compound 1 as compared to dihydrexidrine (DHX) measured in terms of rotations (360 CCW) over time (minutes). Both drugs were administered 1 mg/kg subcutaneously (SC), and the number of rotations measured (i.e., rotations counter clockwise) over time (minutes) using the model first described by

Ungerstedt (Ungerstedt and Arbuthnott, 1970).

FIG. 2 is a graph showing that Compound 1 is a full dopamine D₁ receptor agonist. In FIG. 2, Compound 1 is compared to dihydrexidine in their ability to activate adenylate cyclase (shown as a percentage of activation caused by 100 μM dopamine vs. the log concentration for Compound 1 or dihydrexidine).

### DETAILED DESCRIPTION

The present invention relates to compounds which are either selective dopamine D₁ agonists, or which have activity at both the D₁ and D₂ receptor subtypes, as well as compositions including the compounds, and methods of treatment using the compounds.

Definitions:

The term “C₁-C₄ alkyl” as used herein refers to branched or straight chain alkyl groups comprising one to four carbon atoms, including, but not limited to, methyl, ethyl, propyl, isopropyl, n-butyl, t-butyl and cyclopropylmethyl.

The term “pharmaceutically acceptable salts” refers to those salts which are suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared according to conventional methods in situ during the final isolation and purification of the compounds, or separately by reacting the free base with a suitable organic acid.

The term “phenoxy protecting group” as used herein refers to substituents on the phenolic oxygen which prevent undesired reactions and degradations during synthesis and which can be removed later without effect on other functional groups on the molecule. Such protecting groups and the methods for their application and removal are well known in the art. They include ethers, such as methyl, isopropyl, t-butyl, cyclopropylmethyl, cyclohexyl, allyl ethers and the like; alkoxyalkyl ethers such as methoxymethyl or methoxyethoxymethyl ethers and the like; alkylthioalkyl ethers such a methylthiomethyl ethers; tetrahydropyranyl ethers; arylalkyl ethers such as benzyl, o-nitrobenzyl, p-methoxybenzyl, 9-anthrylmethyl, 4-picolyl ethers and the like; trialkylsilyl ethers such as trimethylsilyl, triethylsilyl, t-butyldimethylsilyl, t-butyldiphenylsilyl ethers and the like; alkyl and aryl esters such as acetates, propionates, n-butyrates, isobutyrates, trimethylacetates, benzoates and the like; carbonates such as methyl, ethyl, 2,2,2-trichloroethyl, 2-trimethylsilylethyl, vinyl, benzyl and the like; and carbamates such as methyl, isobutyl, phenyl, benzyl, dimethyl and the like.

The term “catechol-protecting groups” refers to groups used to derivatize catechol hydroxyl oxygen atoms in order to prevent undesired reactions or degradation during a synthesis (c.f., T. H. Greene, Protective Groups in organic Synthesis, John Wiley & Sons, Inc., Third Edition, 1999). These derivatizing groups may be selected from phenol-protecting groups or they may be selected from those groups which are particularly suitable for the protection of catechols because of the proximity of the two hydroxyl functions on the catechol ring. Commonly used catechol-protecting groups include dimethyl ethers, dibenzyl ethers, cyclohexylidene ketals, methylene acetals, acetonide derivatives, diphenylmethylene ketals, cyclic borate esters, cyclic carbonate esters, cyclic carbamates, and the like.

The term “C₁-C₄ alkoxy” as used herein refers to branched or straight chain alkyl groups comprising one to four carbon atoms bonded through an oxygen atom, including, but not limited to, methoxy, ethoxy and t-butoxy.

As used herein, an “agonist” is a substance that binds to its receptor target, in this case, the dopamine D₁ receptor, or the dopamine D₁ and D₂ receptors, and causes a functional effect of the same character as caused by the natural ligand for that receptor, in this case dopamine. As used herein, an “antagonist” is a substance that binds to its target receptor, in this case, the dopamine D₁ receptor, or the dopamine D₁ and D₂ receptors, and causes no effect by itself but blocks the actions of dopamine or a dopamine agonist that might be present. As used herein, a “partial agonist” is a substance that binds to its target receptor, and no matter how high the concentration, causes a functional effect that is intermediate between that caused by a full agonist and an antagonist. The term “intrinsic activity” or “efficacy” as used herein relates to the measure of biological action caused in a particular assay system. In some circumstances, intrinsic activity may vary depending on the particular second messenger system involved (see Hoyer and Boddeke, 1993; Mailman, 2007; Urban et al., 2007). In addition, some drugs can have markedly different intrinsic activity when one measures different signaling pathways mediated by the same receptor. This phenomenon is most commonly termed “functional selectivity” (Mailman, 2007; Mailman and Gay, 2004; Mailman and Huang, 2007; Urban et al., 2007). Where such contextually specific evaluations are relevant, and how they might be relevant in the context of the present invention, will be apparent to one of ordinary skill in the art.

I. Compounds

The compounds include novel C₂, C₃, and/or C₄ -substituted trans-5,6,6a, 7,8,12b-hexahydrobenzo[a]phenanthridines, oxa-, thio, and azo substituted analogs thereof, prodrugs or metabolites of these compounds, and pharmaceutically acceptable salts thereof.

The compounds can bind to, and either specifically modulate dopamine D₁ receptors, or modulate both dopamine D₁ and D₂ receptors, in the patient's brain in what are termed mesocortical, mesolimbic, nigrostriatal, and tuberoinfundibular dopamine terminal fields. When so bound, the compounds may affect the signaling of dopamine receptors in both pre- and postsynaptic cells.

Receptor binding constants are a quantitative measure of the ability of a compound to bind to its target receptor(s). See, for example, Cheng and Prusoff (,1973). The receptor binding constants for the D₁ receptor of the compounds described herein generally exceed about 0.1 nM, often exceed about 1 nM, and frequently exceed about 10 nM, but are always less than about 1 μM. Preferred compounds generally have receptor binding constants for the D₁ receptor less than about 1 μM, and can be less than about 100 nM.

The compounds can cross the blood-brain barrier, and thus enter the central nervous system of the patient. Log P values provide a measure of the ability of a compound to pass across a diffusion barrier, such as a biological membrane, including the blood brain barrier (Hansch et al., 1995). Typical log P values for the compounds described herein are generally greater than about −0.5, often are greater than about 0, and frequently are greater than about 0.5, and are typically less than about 3, often are less than about 2, and frequently are less than about 1.

In one embodiment, the compounds have the structure represented by Formula 1 below:

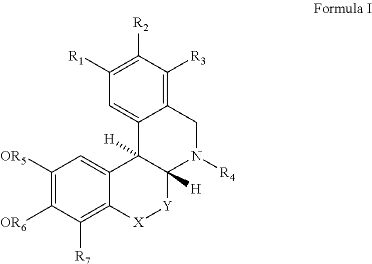

wherein

R₁ is selected from the group consisting of C₁₋₃ alkyl, C₂₋₃ alkenyl or C₂₋₃ alkynyl, alkanoyl moieties having 1 to 3 carbon atoms, halo-,

R₂ is H or C₁₋₄ alkyl;

R₃ is H, Cᵢ₋₇ alkyl, C₃₋₇ cycloalkyl, C₃₋₆ alkenyl, C₃₋₆ alkynyl, Cᵢ₋₇ alkanoyl, arylalkyl, arylalkanoyl having 1 to 3 carbon atoms in the alkyl portion of the moiety, wherein the aryl ring can be substituted by fluorine, chlorine or bromine atoms;

R₄ is selected from the group consisting of H, C₁₋₃ alkyl, C₂₋₃ alkenyl, and C₂-₃ alkynyl;

R₅ and R₆ are, independently, selected from the group consisting of H or hydroxyl protecting groups.

R₇ are, independently, selected from methyl, ethyl, hydrogen or halo.

X and Y are, independently, C(R₈)₂, oxygen, sulfur, or NR₇, where R₇ is selected from the group consisting of H, amine protecting groups, C₁₋₇ alkyl, C₃₋₇ cycloalkyl, alkylaryl, and arylalkyl and R₈ is selected from the group consisting of H, C₁₋₇ alkyl, C₃₋₇ cycloalkyl, alkylaryl, and arylalkyl, with the proviso that at least one of X or Y must be C(R₈)₂, or a pharmaceutically acceptable salt thereof.

One specific compound is shown below, referred to herein as Compound 1:

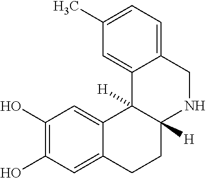

This compound is a selective D₁ agonist.

The compounds may have asymmetric centers and occur as racemates, racemic mixtures, individual diastereomers or enantiomers, with all isomeric forms being included in the present invention. Those compounds having a chiral center may exist in and be isolated in optically active and racemic forms. Some compounds may exhibit polymorphism. The present invention encompasses racemic, optically-active, polymorphic, or stereoisomeric forms, and mixtures thereof, of the compounds described herein, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Space-filling representations of the low energy conformations for (+)-trans-10,11-dihydroxy-5,6,6a,7,8,12b-hexahydrobenzo[a]phenanthridine [(+)-dihydrexidine] and a variety of other high intrinsic activity full D₁ agonists have been compared (Mottola et al., 1996). Based on the underlying model of the D₁ pharmacophore, it is anticipated that both the affinity and intrinsic activity of racemic Compound 1 (and substituted analogs thereof, as described herein) reside in only one of its enantiomers—the 6aR, 12bS absolute configuration (and its homochiral analogs). Resolution of the racemate using art recognized separation techniques is expected to yield one Compound 1 isomer with approximately twice the D₁ affinity exhibited by racemic Compound 1, thus making its affinity for the D₁ receptor similar to (+)-dihydrexidine. It also has been shown that the D₂ properties of dihydrexidine reside in the same enantiomer (i.e., 6aR, 12bS) that is the high affinity full agonist at the D₁ receptor. On this basis, it is expected that both the D₁ and D₂ properties of Compound 1 will also reside in the homochiral enantiomer. Thus, in addition to the importance of the actions at D₁ receptors, the optical isomers of Compound 1 and appropriate analogs may constitute significant tools to study the phenomena of “functional selectivity” (Mailman, 2007; Urban et al., 2007).

Compounds, pharmaceutical compositions including the compounds, and methods of treatment, in which the compounds are enantiomerically-enriched in the 6aR, 12bS enantiomer, are also within the scope of the invention. In one aspect of this embodiment, the compounds are in an enantiomeric mixture in which the desired enantiomer is at least 95%, 98% or 99% free of the other enantiomer.

The compounds are dopamine receptor ligands with unexpectedly longer duration of action than the parent ligands of this class. The longer duration of action caused by the 2-position substituents can be used in concert with substitution at other positions to create novel ligands with unexpected pharmacokinetic and pharmacodynamic properties. Ideally, the duration of action is sufficient for administration no more than three times daily. Typically, the compounds cause typical or functionally-selective activation of one of more dopamine receptors.

The differences between the claimed compounds, and those described in the prior art, can be more clearly seen with reference to the following structures. As shown below, the compound Compound 1 differs from dihydrexidine by the presence of a methyl group in position R₁.

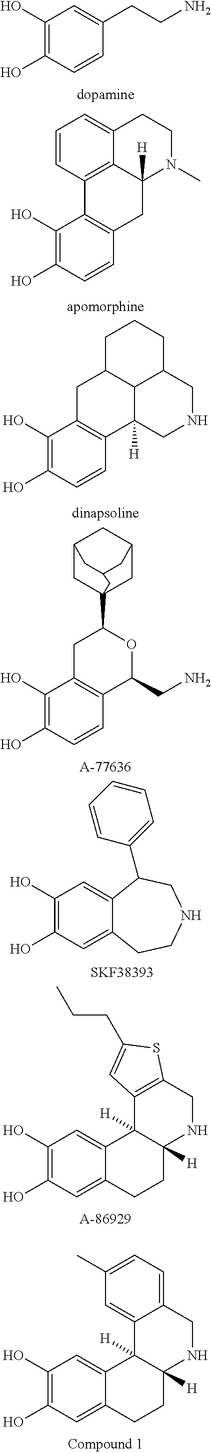

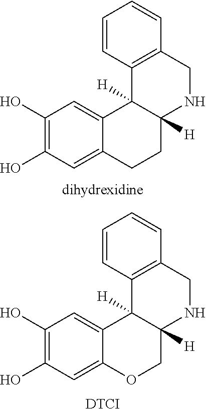

### Structures of Compound 1 and Other Prototypical D₁ Agonists.

II. Methods of Preparing the Compounds

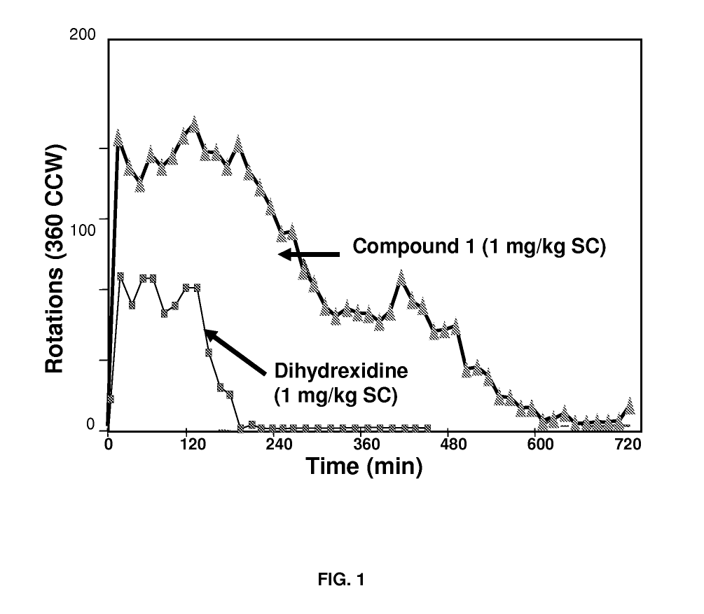

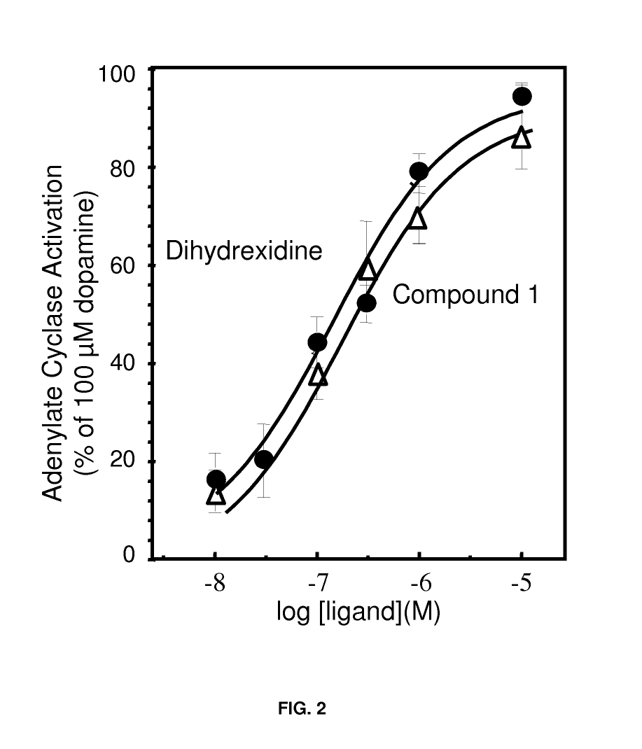

The compounds of this invention are prepared using the same preparative chemical steps described for the preparation of the hexahydrobenzo[a]phenanthridine compounds described and claimed in U.S. Pat. No. 5,047,536, issued Sep. 10, 1991, which is expressly incorporated herein by reference. The present compounds can be prepared using the chemical reactions depicted in the reaction scheme illustrated in FIGS. 1 and 2 of U.S. Pat. No. 5,047,536 using the appropriately substituted benzoic acid acylating agent starting material instead of the benzoyl chloride reagent used in the initial reaction step. Thus, for example, use of 4-methylbenzoyl chloride will yield a 2-methyl hexahydrobenzo[a]phenanthridine compound.

Embodiments of general reaction schemes are shown below as Scheme I.

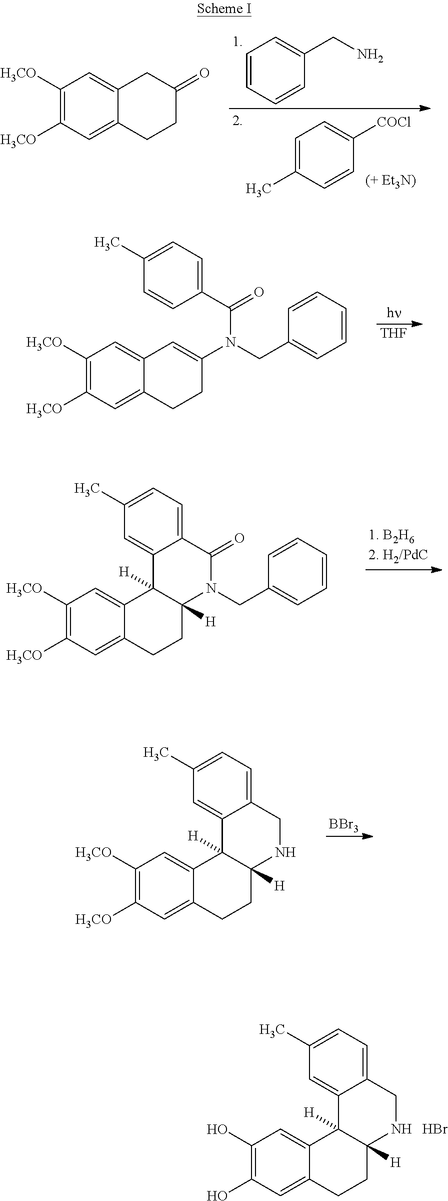

As shown in Scheme I, a 6,7,-difunctionalized-beta-tetralone is reacted with benzylamine in a suitable solvent, such as toluene, to provide the benzyl imine analogue. To arrive at the compounds described herein, the tetralone is suitably functionalized at the 6 and 7 positions with OR₅ and OR₆ substituents, or protected versions thereof. In Scheme I, the OR₅ and OR₆ substituents are initially OCH₃ groups, which are ultimately deprotected to form OH groups. Other protecting groups can be used, and suitable hydroxyl protecting groups are described, for example, in Greene and Wuts, Protective Groups in Organic Synthesis, Wiley-Interscience, New York, 1999.

A tertiary amine such as triethylene amine, or other suitable acid scavenger, is added, and a suitably functionalized (and suitably protected), activated benzoic acid is reacted with the benzyl imine to form an enamide, which retains the benzyl protecting group. One representative benzoic acid, and the one used in Scheme I, is 4-methyl-benzoic acid.

The activated benzoic acid moiety can be an acid halide (as shown below in Scheme I), such as a suitably functionalized benzoyl chloride, or an acid anhydride, or other groups known to readily form enamides upon reaction with an enamine. Alternatively, traditional coupling chemistry involving a coupling agent such as DCC (N,N-dicyclohexylcarbodiimide) and the free acid and free base can be used.

Irradiation of the resulting benzoic amide forms a lactam. The irradiation can be performed, for example, in an Ace Glass 250 ml photochemical reactor, over a period of several hours using, for example, a 450 watt Hanovia medium pressure, quartz, mercury-vapor lamp seated in a water cooled, quartz immersion well.

The lactam can be reacted with BH₃ etherate (for example, in THF or diethyl ether) to reduce the lactam to a cyclic amine. This cyclic amine can be de-benzylated by reaction with hydrogen and a suitable catalyst, such as a 10% palladium on carbon (Pd—C).

If it is desirable that OR₅ or OR₆ be an OH group, then one can start with an OCH₃ group, then deprotect, for example, by reaction with BBr₃.

The starting material, 6,7-dimethoxy-2-tetralone, is readily available using the facile synthetic method described by Qandil et al. (,1999). The photochemical cyclization reaction results in relatively high yields. The photochemical reaction precursor, the enamide, can be prepared in large amounts and then photocyclized in a number of gram batches.

Alternate approaches to the synthesis of the compounds described herein can be used. For example, Asano et al. (,2001) discloses an asymmetric synthesis of dihydrexidine that involved three main steps, external chiral ligand-controlled conjugate addition of phenyllithium, Curtius conversion of a carboxylic group to an amino group, and finally Pictet-Spengler type cyclization completing the skeleton construction. This approach can be modified such that the unsubstituted aromatic ring in dihydrexidine includes the substituents described herein.

Negash and Nichols (2001) described a process for preparing dihydrexidine, using as a key step the cyclization of an acid chloride intermediate, via decarbonylation, to the hexahydrobenzo[a]phenanthridine. This approach can also be modified, with appropriate substitution of starting materials, to arrive at the compounds described herein, as shown below.

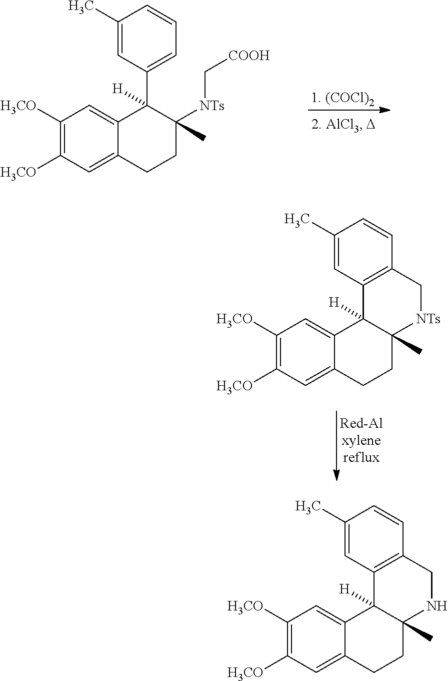

III. Pharmaceutical Compositions

The compounds described herein can be formulated in conventional drug dosage forms. Preferred doses of the present compounds depend on many factors, including the indication being treated, the route of administration, and the overall condition of the patient. For oral administration, for example, effective doses of the present compounds are expected to range from about 0.1 to about 25 mg/kg, more typically about 0.5 to about 5 mg/kg. Effective parenteral doses can range from about 0.01 to about 5 mg/kg of body weight, more typically from about 0.1 to about 1 mg/kg of body weight. In general, treatment regimens utilizing compounds in accordance with the present invention comprise administration of from about 1 mg to about 500 mg of the compounds of this invention per day in multiple doses or in a single dose.

Liquid Dosage Forms

Liquid dosage forms for oral administration may include pharmaceutically acceptable emulsions, microemulsions, solutions, suspensions, and syrups containing inert diluents commonly used in the art, such as water. Such compositions may also comprise adjuvants such as wetting agents, emulsifying and suspending agents, sweetening, and flavoring agents. Injectable preparations of the compounds of the present invention can be formulated utilizing art-recognized procedures by dispersing or dissolving an effective dose of the compound in a parenterally acceptable diluent such as water, or more preferably isotonic sodium chloride solution. The parenteral formulations can be sterilized using art-recognized microfiltration techniques.

Solid Dosage Forms

The compounds of this invention can also be formulated as solid dosage forms for oral administration such as capsules, tablets, powders, pills and the like. Typically the active compound is admixed with an inert diluent or carrier such as sugar or starch and other excipients appropriate for the dosage form. Thus, tablet formulations will include acceptable lubricants, binders and/or disintegrants. Optionally powder compositions comprising an active compound of this invention and, for example, a starch or sugar carrier can be filled into gelatin capsules for oral administration.

Other dosage forms of the compounds of the present invention can be formulated using art-recognized techniques in forms adapted for the specific mode of administration. This can include novel formulations such as Zydis, which is an oral disintegrating tablet including gelatin, mannitol, sodium methyl paraben, and sodium propyl paraben, and other oral disintegrating tablets.

Nanoparticulate delivery systems can also be used. Nanoparticulate compositions, first described in U.S. Pat. No. 5,145,684, include particles comprising a therapeutic agent having adsorbed onto the surface thereof a non-crosslinked surface stabilizer.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. No. 5,399,363 and U.S. Pat. No. 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(-) Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate,” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” all of which are specifically incorporated by reference. In addition, U.S. patent application No. 20020012675 A1, published on Jan. 31, 2002, for “Controlled Release Nanoparticulate Compositions,” describes nanoparticulate compositions, and is specifically incorporated by reference.

Transdermal Formulations

In some embodiments, the compositions are present in the form of transdermal formulations, such as that used in the FDA-approved agonist rotigitine transdermal (Neupro patch). Another suitable formulation is that described in U.S. Publication No. 20080050424, entitled “Transdermal Therapeutic System for Treating Parkinsonism.” This formulation includes a silicone or acrylate-based adhesive, and can include an additive having increased solubility for the active substance, in an amount effective to increase dissolving capacity of the matrix for the active substance.

The transdermal formulations can be single-phase matrices that include a backing layer, an active substance-containing self-adhesive matrix, and a protective film to be removed prior to use. More complicated embodiments contain multiple-layer matrices that may also contain non-adhesive layers and control membranes. If a polyacrylate adhesive is used, it can be crosslinked with multivalent metal ions such as zinc, calcium, aluminum, or titanium ions, such as aluminum acetylacetonate and titanium acetylacetonate.

When silicone adhesives are used, they are typically polydimethylsiloxanes. However, other organic residues such as, for example, ethyl groups or phenyl groups may in principle be present instead of the methyl groups. Because the active compounds are amines, it may be advantageous to use amine-resistant adhesives. Representative amine-resistant adhesives are described, for example, in EP 0 180 377.

Representative acrylate-based polymer adhesives include acrylic acid, acrylamide, hexylacrylate, 2-ethylhexylacrylate, hydroxyethylacrylate, octylacrylate, butylacrylate, methylacrylate, glycidylacrylate, methacrylic acid, methacrylamide, hexylmethacrylate, 2-ethylhexylmethacrylate, octylmethacrylate, methylmethacrylate, glycidylmethacrylate, vinylacetate, vinylpyrrolidone, and combinations thereof.

The adhesive must have a suitable dissolving capacity for the active substance, and the active substance most be able to move within the matrix, and be able to cross through the contact surface to the skin. Those of skill in the art can readily formulate a transdermal formulation with appropriate transdermal transport of the active substance.

Certain pharmaceutically acceptable salts tend to be more preferred for use in transdermal formulations, because they can help the active substance pass the barrier of the stratum corneum. Examples include fatty acid salts, such as stearic acid and oleic acid salts. Oleate and stearate salts are relatively lipophilic, and can even act as a permeation enhancer in the skin.

Permeation enhancers can also be used. Representative permeation enhancers include fatty alcohols, fatty acids, fatty acid esters, fatty acid amides, glycerol or its fatty acid esters, N-methylpyrrolidone, terpenes such as limonene, alpha-pinene, alpha-terpineol, carvone, carveol, limonene oxide, pinene oxide, and 1,8-eucalyptol.

The patches can generally be prepared by dissolving or suspending the active agent in ethanol or in another suitable organic solvent, then adding the adhesive solution with stirring. Additional auxiliary substances can be added either to the adhesive solution, the active substance solution or to the active substance-containing adhesive solution. The solution can then be coated onto a suitable sheet, the solvents removed, a backing layer laminated onto the matrix layer, and patches punched out of the total laminate.

Inhalable Formulations

In one embodiment, the compounds are administered via inhalable (i.e., pulmonary and intranasal) formulations. These can be particularly useful for treating patients suffering from Parkinson's Disease, where later stage patients have trouble swallowing.

In another embodiment, the present invention relates to a method of administering the compounds described herein to a patient, comprising administering to the patient a therapeutically effective amount of the pulmonary or intranasal compositions described herein.

Intranasal Formulations

In one embodiment, the present invention relates to an intranasal pharmaceutical formulation containing a pharmaceutically acceptable salt of the compounds described herein. Such intranasal formulations are useful for treating disorders where the administration of the compounds is beneficial, in particular in the treatment of Parkinson's Disease, and with patients who have difficulty swallowing orally-administered formulations.

Ideally, the formulations have no detectable microbiological contamination. In one embodiment, this is achieved without using potentially irritating preservatives, such as ethanol or benzalkonium chloride, even though such preservatives can be added as needed. Ideally, the formulations produce low irritation of the nasal mucosa, and avoid nasal vestibulitis, when administered.

In one aspect of this embodiment, the liquid intranasal pharmaceutical formulation includes a pharmaceutically-acceptable acid addition salt of the compounds described herein, and a cyclodextrine, such as alpha-or beta-cyclodextrin, or methylated versions thereof. Representative pharmaceutically acceptable acid addition salts are described herein, and specifically include hydrochloride, citrate and methanesulfonate.

The liquid intranasal formulation can further include buffer salts, e.g. phosphates or acetates, and as such may be present as a buffered aqueous solution, for example, phosphate buffered saline (PBS).

The intranasal formulation can also further include a viscosity-enhancing substance. Glycerol and carboxymethylcellulose (CMC) are non-limiting examples of such viscosity enhancers. Glycerol can be particularly preferred, as it also has a soothing effect on the nasal mucosa.

In one aspect of this embodiment, the viscosity of the intranasal formulations is between 0.8 and 1.5 mm²/s, for example, around 1.2 mm²/s. The viscosity can be determined, for example, by an Ubbelohde capillary viscosimeter with suspending ball-level for the determination of kinematic viscosity according to DIN 51562, part 1.

The pH-value of the intranasal formulations is ideally in the range of 4.5 to 6.5, more preferably around 5.8+/−0.3. This pH range can provide an optimum between good drug stability and solubility, and good flux across the nasal mucosal membrane (which tends to be better at around pH 7). The pH value of the intranasal formulation can be adjusted during or after its preparation with a pharmaceutically acceptable acid or base, for example, citric acid or a citrate salt.

In one aspect, the intranasal formulation does not contain a further absorption enhancer, preservative and/or antioxidant. In other aspects, the intranasal formulations contain further absorption enhancers. Such enhancers include, but are not limited to, surfactants and/or emulsifiers, particularly non-ionic surfactants such as TWEEN 80™ or cremophor RH40™. Representative antioxidants include ascorbates or sorbates. Representative preservatives include, but are not limited to, antimicrobial substances such benzalkonium chloride.

In one aspect of the invention, a cyclodextrin, such as alpha-cyclodextrin or beta-cyclodextrin, and methylated versions thereof, is present. The cyclodextrins can increase the storage stability of the intranasal formulations. The concentration of the cyclodextrin in solution need not exceed 0.5 g/ml, and a suitable range is typically between about 0.001 and about 0.1 g/ml, more preferably between 0.05 and 0.1 g/ml and most preferably between 0.08 and 0.09 g/ml. The use of cyclodextrins in intranasal drug delivery is described, for example, in Merkus et al., Advanced Drug Delivery Reviews 36 (1999) 41-57.

In one embodiment, the active compound is present in the intranasal formulation at a concentration of between about 1 and about 6 mg/ml, in one aspect, in an aqueous buffered solution. In a further and independent aspect, the intranasal formulation contains between 0.03 and 0.1 g/ml of a cyclodextrin.

To evaluate the storage stability of potential nasal formulations, one can measure the concentration of the active over time, for example, using gradient HPLC analysis.

Pulmonary Formulations

The compounds described herein can be well-absorbed when administered into the airways, particularly due to the large absorptive area, low enzymatic activity, and near-neutral pH in the lung.

The compounds described herein can be delivered, for example, by means of metered dose inhalation (MDI) devices, in which a physiologically inert propellant of high vapor pressure is used to discharge a precise amount of medication with each operation. These MDI devices, also known as aerosols or inhalers, have found widespread use among patients suffering, for example, from episodic or chronic asthma. The propellants of choice have historically been chlorofluoro-carbons, such Propellant 11 (trichlorofluoromethane), Propellant 12 (dichlorodifluoromethane) and Propellant 114 (dichlorotetrafluoroethane).

Alternative propellant vehicles include two—HFC-134a (1,1,1,2-tetrafluoroethane) and HFC-227ea (1,1,1,2,3,3,3-heptafluoropropane).

Ideally, the compounds are not immiscible with or insoluble in, and therefore incompatible with, the propellants. Surfactants can be used to prevent aggregation (in the form of “caking” or crystallization, for example) of the compounds in the reservoir of the inhaler, to facilitate uniform dosing upon aerosol administration, and to provide an aerosol spray discharge having a favorable respirable fraction (that is, a particle size distribution such that a large portion of the discharge reaches the alveoli where absorption takes place, and thus produces high lung deposition efficiencies).

Representative pulmonary formulations and devices (i.e., inhalers) are described, for example, in U.S. Pat. No. 5,225,183. The formulations included HFC-134a, a surface active agent, and an adjuvant or co-solvent having a higher polarity than HFC-134a Representative adjuvants or co-solvents having a higher polarity than HFC-134a include alcohols such as ethanol, isopropanol and propylene glycol; hydrocarbons such as propane, butane, isobutane, pentane, isopentane and neopentane; and other propellants such as Propellants 11, 12, 114, 113 and 142b. The adjuvant purportedly provides a propellant system having comparable properties to those based on CFC propellants and therefore allow the use of traditional surfactants. Blends of HFC-134a with other solvents or propellants including dimethyl ether; fluorocarbons such as perfluoropropane, perfluorobutane and perfluoropentane; and hydrochlorofluorocarbons such as HCFC-123 are disclosed in U.S. Pat. No. 5,190,029.

Polar surfactants such as polyethylene glycol, diethylene glycol monoethyl ether, polyoxyethylene (20) sorbitan monooleate, propoxylated polyethylene glycol, and polyoxyethylene (4) lauryl ether can be used, as is disclosed in U.S. Pat. No. 5,492,688.

U.S. Pat. No. 5,182,097 discloses that HFC-134a can be used as the sole propellant if oleic acid is used as the surfactant. U.S. Pat. No. 5,182,097 discloses that using fluorinated surfactants allows the HFC-134a as the sole propellant. PCT Application No. WO 91/11173 discloses that mixtures of fluorinated surfactants with conventional surfactants or other adjuvants such as polxamers or polyethylene glycols allow the use of hydrofluorocarbon propellants. Non conventional excipients which have been used to prepare aerosol formulations with halogenated alkane propellants include protective colloids, see PCT Application No. WO 95/15151, and tocopherol, see PCT Application No. WO 95/24892.

In addition to delivery via metered dose inhalers, other pulmonary delivery systems include powders, microparticles and aqueous and non-aqueous based solutions or dispersions which are administered through and/or into the airways by nasal or trachael routes.

Accordingly, the present invention provides a method of administering the compounds described herein to a patient, comprising administering a therapeutically effective amount of the compounds described herein to the airways of the patient. This deliver means can occur through nasal or tracheal administration and can be in the form of a formulation or composition comprising a compound delivered in the form of a solid, microparticle or powder, and can further comprise a pulmonary delivery excipient selected from solids or liquids which are aqueous based or non-aqueous based. Liquid formulations delivered through the airways can be prepared in aqueous or non-aqueous vehicles, and delivered to the airways by means of drops or sprays.

Thus, in one embodiment, the present invention relates to a composition for pulmonary delivery comprising a compound described herein dispersed in an aqueous or non-aqueous delivery vehicle. The aqueous vehicle is selected from pure water, substantially pure water or water combined with other excipients such as salts, ions or other excipients which are generally used in aqueous based systems. The liquid formulations are in the form of solution based dispersions or solutions in solvents or cosolvents such as alcohols or glycols with water. Non-aqueous solutions include those alchohol or glycol based systems which may have some water, but which are not comprised of a majority percentage of water and which are known to those of skill in the art as effective and safe delivery vehicles. Non-aqueous solutions also include those systems containing halogenated hydrocarbons. Administration of liquid formulations in the form of drops or dispersions occurs through the nose and/or trachea to facilitate absorption of the formulation and prodrug and/or active ingredients into the lungs and ultimately delivery to the dopamine receptors where the medicinal effect is achieved to treat, for example, Parkinson's disease. Devices can be used to assist in the delivery of the active agent(s).

In another embodiment, the present invention provides a pharmaceutical composition for aerosol delivery of a compound described herein, which include, in addition to the active agent, a propellant, poloxamer and tocopherol.

Numerous chlorofluorocarbon (CFC) and non-chlorofluorocarbon (NCFC) aerosol propellants can be used. Representative CFC propellants include CFC-11 (trifluorochloromethane), CFC-12 (dichlorodifluoromethane) and CFC-114 (dichlorotetrafluoroethane). Ideally, the propellants are non-ozone depleting halogenated alkanes such as HCFC-123 (1,1,1-trifluoro-2,2-dichloroethane), HCFC-124 (1,1,1,2-tetrafluorochloroethane), HCFC-141b, HCFC-225, HFC- 125, FC-C51-12 (perfluorodimethylcyclobutane), DYMEL A (dimethyl ether), DYMEL 152a (1,1-difluoroethane), HFC-134a, and HFC-27ea.

The poloxamers that can be used in the compositions are block copolymers of ethylene oxide and propylene oxide. The poloxamers typically have a molecular weight of from about 1950 to about 3350 and a hydrophilic lipophilic balance (hlb) of from about 10 to about 20. Representative poloxamers include poloxamer 124 (Pluronic® L44, MW about 2200, hlb 16), Pluronic® 10R5 (MW about 1950, hlb 15), Pluronic® 17R4 (MW about 2650, hlb 12), Pluronic® 22R4 (MW about 3350, hlb 10) and Pluronic® L64 (MW about 2900, hlb 15), all available from BASF Corp., Parsippany, N.J.

Poloxamer can be present in a concentration of from about 0.001% to about 5%, preferably in a concentration of from about 0.01% to about 2% and most preferably in a concentration of from about 0.1% to about 1%. Preferred poloxamers have a molecular weight of from about 1950 to about 2900 and an hlb of from about 12 to about 16. The most preferred poloxamer of the present invention is poloxamer 124.

Poloxamer 124 has the chemical name .alpha.-hydro-.omega.hydroxypoly(oxyethylene)poly(oxypropylene)poly(oxyeth ylene) block copolymer. As listed in USPNF XVII, poloxamer 124 has a molecular weight of between 2090 and 2360 and a hlb of 16. It is a liquid at ambient temperature and has weight percent oxyethylene of 46.7%.+-.1.9% and unsaturation (mEq/g) of 0.020.+-.0.008 see Wade, A. and Weller P. L., eds., Handbook of Pharmacetucial Excipients, (2 ed., Washington, D.C.: American Pharmaceutical Assoc.) 1994, 352-354. Pluronic® L44 has a molecular weight of about 2250.

The aerosol compositions can also contain additional inactive excipients such as antioxidants and flavoring and/or taste masking agents to stabilize the drug and improve dosimetry. Preferred antioxidants are tocopherol derivatives such as d-alpha tocopherol, dl-alpha tocopherol, d-alpha tocopherol acetate, dl-alpha tocopherol acetate d-alpha tocopherol acid succinate and dl-alpha tocopherol acid succinate. The most preferred antioxidant is dl-alpha tocopherol acetate. The antioxidant may be present in a concentration of from about 0.001% to about 5%, preferably in a concentration of from about 0.01% to about 2% and most preferably in a concentration of from about 0.01% to about 1%.

A sweetener such as aspartame and/or a taste masking agent such as menthol may also be present in concentrations of between about 0.001% and and about 10% by weight, preferably in a concentration of between about 0.002% and about 5% by weight and more preferably in a concentration of between about 0.01% and 1%.

The MDI compositions can be prepared by combining poloxamer and any other excipients with a medicament which has been milled or otherwise reduced to a desired particle size, and placing the mixture in a suitable aerosol container or vial. After sealing the container, an aerosol propellant is introduced and the system is agitated to fully blend the ingredients. In some instances, it may be necessary to wet-mill the medicament in a closed system, as for example under temperature and pressure conditions which permit the medicament to be milled while mixed with a liquid-phase aerosol propellant. It is expected that, for any particular combination of medicament, propellant and poloxamer, the ideal order of addition of ingredients and the conditions under which they are to be combined may readily be determined.

Uniformity of MDI Delivery

Delivery uniformity of the MDI compositions can be tested, for example, as follows: An aerosol container can be shaken and it valve primed by aerosolizing 5 times in succession. After priming, the aerosol container can be shaken and then attached to an atomizing nozzle which can be cut from an actuator. With the nozzle pointed downward, the canister can be placed into a 30-mL beaker containing 10 mL of methanol until the nozzle touches the bottom of the beaker. Then, a total of 2 sprays, each separated by a 5 second pause, can be delivered into the beaker. The valve stem and ferrule can be rinsed with acetonitrile. The amount of drug in each sample can be analyzed, for example, by HPLC.

Aerosol Particle Size Distribution

Particle size data in an aerosol formulation can be determined, for example, using the Malvern laser diffraction particle sizer (Model 2600C). Samples can be analyzed as aerosolized aprays in air. An aerosol can with an actuator assembly can be mounted on a clamp stand so that the spray jet is around 12.5 cm from the laser beam. Beam length, i.e., the length of aerosol flume along the path of the laser bean, can be about 10 cm. In this configuration, the distance of the objective lens can be 3 cm from the middle of the aerosol flume, and the IR beam of the spray synthronizer can be 4 cm from the spray jet. Also, the laser beam and the IR beam can be parallel and approximately 8.5 cm apart. A representative number of sprays can be actuated and analyzed individually assuming a log-normal distribution model. The detection of spray duration is approximately 15 milliseconds (ms), i.e., beginning from 70 ms and ending at 85 ms after interruption of the IR beam by the aerosol.

The particle size of aerosolized product determines the extent as well as the pattern of drug deposition in the respiratory tract. Ideally, the emitted particle size is less than 10 microns, preferably less than around 5 microns.

Bioavailability of MDI Compositions

The bioavailability of the formulations can be assessed, for example, using a non-crossover bioavailability study involving a suspension aerosol formulation and an iv injection solution. The aerosol sprays can be delivered anteriorly via a tracheal stoma in a test animal. Plasma concentration profiles of the active metabolite of the compounds following administration of the formulations can then be compared, and ideally demonstrate that lung absorption of the compound following inhalation delivery occurs at least as efficiently as I.V. administration, or, at a minimum, at sufficient levels to achieve a desired physiological effect.

Optional Additional Components

The pharmaceutical composition also can include various other components as additives or adjuncts. Exemplary pharmaceutically acceptable components or adjuncts which are employed in relevant circumstances include antioxidants, free radical scavenging agents, other centrally acting drugs, peptides, growth factors, antibiotics, bacteriostatic agents, immunosuppressives, anticoagulants, buffering agents, antiinflammatory agents, antipyretics, time release binders, anesthetics, steroids and corticosteroids. Such components can provide additional therapeutic benefit, act to affect the therapeutic action of the pharmaceutical composition, or act towards preventing any potential side effects which may be posed as a result of administration of the pharmaceutical composition. In certain circumstances, a compound of the present invention can be employed as part of a pharmaceutical composition with other compounds intended to prevent or treat a particular disorder.

IV. Methods of Treatment

The compounds described herein can be used to treat or prevent a variety of disorders mediated by dopamine neurons or dopamine-target neurons. The methods involve administering to a patient an amount of a compound effective for providing some degree of prevention of the progression, amelioration of the symptoms, or amelioration of the reoccurrence, of a disorder mediated by dopamine systems, for example, neurologic or psychiatric brain disorders.

The compounds described herein are useful for treating those types of conditions and disorders for which other types of dopamine agonists have been proposed as therapeutics. Representative CNS disorders that can be treated include Parkinson's disease, parkinsonism, restless leg syndrome, schizophrenia, presenile dementia (early onset Alzheimer's disease), senile dementia (dementia of the Alzheimer's type), and other disorders with cognitive deficits (including age-associated cognitive deficits), substance abuse, tardive dyskinesia, attention deficit hyperactivity disorder, mania, anxiety, Huntington's chorea, Tourette's syndrome, and neurodegeneration resulting from acute events like stroke, renal dysfunction, and lung conditions.

In addition, the compounds can be used to improve the cognitive function of “normal” patients, i.e., those who do not manifest clinical signs of cognitive deficit. Thus, for example, “normal” individuals using the compounds described herein, may evidence improvement in memory, cognition, and/or concentration.

The effective dose can vary, depending upon factors such as the condition of the patient, the severity of the symptoms of the disorder, and the manner in which the pharmaceutical composition is administered. Effective doses of the present compounds depend on many factors, including the indication being treated, the route of administration, and the overall condition of the patient. For oral administration, for example, effective doses of the present compounds are expected to range from about 0.1 to about 25 mg/kg, more typically about 0.5 to about 5 mg/kg. Effective parenteral doses can range from about 0.01 to about 5 mg/kg of body weight, more typically from about 0.1 to about 1 mg/kg of body weight. In general, treatment regimens utilizing compounds in accordance with the present invention comprise administration of from about 1 mg to about 500 mg of the compounds described herein per day in multiple doses or in a single dose.

The compounds have the ability to pass across the blood-brain barrier of the patient. As such, such compounds have the ability to enter the central nervous system of the patient. The log P values of typical compounds, which are useful in carrying out the present invention are generally greater than about 0, often are greater than about 0.5, and frequently are greater than about 1. The log P values of such typical compounds generally are less than about 3.5, often are less than about 3, and sometimes are less than about 2.5. Log P values provide an estimate of the ability of a compound to pass across a diffusion barrier, such as a biological membrane (see for example, Hansch et al., 1968).

The compounds, when employed in effective amounts in accordance with the method of the present invention, are selective for the dopamine D₁ receptor, or the dopamine D₁ and D₂ receptors, and do not cause significant activation of receptors associated with undesirable side effects.

The compounds, when employed in effective amounts in accordance with the methods described herein, are effective towards either providing some degree of prevention of disorders, and/or ameliorating the signs and symptoms of these and related CNS disorders. However, such effective amounts of those compounds are not sufficient to elicit side effects to an undesirable level, as is demonstrated by decreased effects on preparations believed to reflect effects on the cardiovascular system. As such, administration of compounds of the present invention provides a therapeutic window in which treatment of certain CNS disorders is provided, and side effects are minimal. That is, an effective dose of the compounds described herein are sufficient to provide the desired effects upon the CNS, but is insufficient (i.e., is not at a high enough level) to provide undesirable side effects at a level that would eliminate the utility of these compounds. Preferably, effective administration of a compound resulting in treatment of CNS disorders occurs upon administration of less than one-half, frequently less than one-fifth, and often less than one-tenth, that amount sufficient to cause side effects that would prevent a compound's clinical use.

Treatment of Parkinson's Disease

The efficacy of selective or non-selective D₂ agonists has largely been disappointing, especially in late-stage, severely disabled PD patients. Dopamine agonists are effective in delaying levodopa-induced dyskinesia in early PD and reducing motor fluctuations in advanced PD, but a recent commentary as levodopa approached its fortieth birthday said “For the foreseeable future, levodopa will remain the gold standard for Parkinson's treatment” (Haughn, 2007). Table 1 summarizes the properties of the available dopamine agonists (noting that many of these are coming off-of-the-market because of cardiac valvuopathy problems). There has been some controversy about the relative importance of the D₂ and D₃ dopamine receptor isoforms as regards the antiparkinson actions of pramipexole and ropinirole. A variety of evidence seems to suggest that it is the D₂, not D₃, properties of these drugs that drives their antiparkinson action (Mailman and Huang, 2007), but except for apomorphine (vide infra), no FDA-approved dopamine agonist comes close to matching a levodopa/adjuvant combination for antiparkinson efficacy.

Currently there are several drugs approved for human use for Parkinson's disease. In the United States this includes pramipexole, ropinirole, rotigitine, and apomorphine. Other ergoline-based molecules (such as pergolide) have either been removed from the market or “black-boxed” because they induce cardiac valve pathology. All of the FDA-approved compounds have highest affinity for D₂ dopamine receptors, and with one exception, they all have but modest efficacy in Parkinson's disease. The exception is apomorphine that is effective as monotherapy, and is the only one of these approved drugs that has high D₁ intrinsic activity. Unfortunately, apomorphine, like dihydrexidine, has a very short duration of action. In addition, its D₂ properties decrease its tolerability (e.g., nausea and emesis).

The compounds described herein have a longer duration of action than dihydrexidine, and those compounds which are D₁-specific increase the tolerability of the compounds (i.e., decrease nausea and emesis relative to apomorphine). Accordingly, they are useful in treating Parkinson's disease.

Treatment of Other CNS Disorders

D₁ receptors are present in high concentration (20 times the density of D₂ receptors) in prefrontal cortex in non-human primates (Lidow et al., 1991), and involved in working memory processes (Murphy et al., 1996; Sawaguchi and Goldman-Rakic, 1994; Williams and Goldman-Rakic, 1995; Zahrt et al., 1997). Optimal stimulation of this brain region is known to potentiate signaling in neurons that are essential to the working memory process (Sawaguchi and Goldman-Rakic, 1991). Lesions of the mesocortical dopamine projection impair working memory performance both in monkeys (Brozoski et al., 1979) and rats (Simon, 1981).

There is strong evidence that D₁ receptor activation may provide cognitive benefits, based on findings that local injection of a D₁ antagonist (but not a D₂ antagonist) into the prefrontal cortex induced deficits in working memory in rhesus monkeys (Sawaguchi and Goldman-Rakic, 1994). D₁ agonists can improve cognitive function both in rodents (Hersi et al., 1995; Steele et al., 1997) and non-human primates (Arnsten et al., 1994; Cai and Arnsten, 1997). Moreover, D₁-like receptors play a critical role in memory processes. Memory dysfunction is associated with defects in late phase long-term potentiation (LTP), which is critical for the persistence of memories (Bach et al., 1999). Several lines of evidence suggest a role for D₁ receptors in this process. Late phase LTP is blocked by D₁-like antagonists (Frey et al., 1991; Huang and Kandel, 1995) and D₁-knockout mice lack late phase LTP altogether (Matthies et al., 1997). Conversely, D₁-like agonists potentiate LTP in the CA1 region of the hippocampus and ameliorate spatial memory deficits in aged mice (Bach et al., 1999; Huang and Kandel, 1995).

In murine models, cognitive-enhancing effects of D₁ agonists can be shown to involve, in part, the release of acetylcholine, and can mimic the effects of galantamine, a cholinesterase inhibitor used clinically for Alzheimer's disease (Di et al., 2007; Steele et al., 1997; Steele et al., 1996)(Steele et al., 1996; Steele et al., 1997; Di et al., 2007). There are numerous other recent reports also showing such benefits of D₁ agonists (Fletcher et al., 2007; Izquierdo et al., 2006; Pezze et al., 2007; Rotaru et al., 2007; Stuchlik, 2007). The behavioral and physiological evidence suggest a normal range of dopamine function in prefrontal cortex that can be described as an “inverted-U” relationship between dopamine transmission and the integrity of working memory (Williams and Castner, 2006). In primate models, there is evidence for the role of D₁ agonists in improving working memory (Arnsten et al., 1994; Cai and Arnsten, 1997; Castner et al., 2000; Schneider et al., 1994). Recently, a preliminary human study provide results consistent with the hypothesis that D₁ agonists have potential in the treatment of cognitive deficits and/or negative symptoms in a variety of conditions including schizophrenia (George et al., 2007; Mu et al., 2007).

Accordingly, the compounds described herein, which are D₁ agonists, can be used to enhance cognition in “normal” patients and treat cognitive disorders in patients suffering therefrom.

The compounds described herein are useful for treating those types of conditions and disorders for which other types of dopamine agonists have been proposed as therapeutics. Representative CNS disorders that can be treated include Parkinson's disease, parkinsonism, restless leg syndrome, schizophrenia, substance abuse, presenile dementia (early onset Alzheimer's disease), senile dementia (dementia of the Alzheimer's type), and other disorders with cognitive or motor deficits, including age-associated cognitive deficits, Huntington's chorea, tardive dyskinesia, hyperkinesia, mania, attention deficit disorder, anxiety, dyslexia, Tourette's syndrome, and neurodegeneration resulting from acute events like stroke, renal dysfunction, and lung conditions.

Treatment of Substance Abuse

It has been hypothesized that most, if not all, drugs that are capable of producing drug-dependence increase dopaminergic transmission in specific brain regions, particularly the nucleus accumbens. Appropriate pharmacological agents can be used to treat substance abuse, including abuse of psychostimulants.

Some D₁ agonists are self-administered by rats (Self et al., 1996b; Self and Stein, 1992) and non-human primates (Weed et al., 1997; Weed et al., 1993; Weed and Woolverton, 1995), and D₁ agonists increased the latency to initiate cocaine self-administration (Caine et al., 1997; Self et al., 1996). However, D₁ agonists do not reinstate non-reinforced responding on a cocaine-paired lever, and in fact decrease the ability of cocaine non-reinforced responding in an animal model of cocaine-seeking (Self et al., 1996). Haney et al. (1999) demonstrated that ABT 431, a selective D₁ receptor agonist with full functional efficacy compared with dopamine, produced significant decreases in the subjective effects of cocaine in a dose-dependent manner, and showed a trend for ABT 431 to decrease cocaine craving.

Accordingly, D₁ agonists such as the compounds described herein can decrease the likelihood that abstinent cocaine abusers in treatment would relapse.

The advantages and features of the invention are further illustrated with reference to the following example, which is not to be construed as in any way limiting the scope of the invention but rather as illustrative of one embodiment of the invention in a specific application thereof.

### EXAMPLE 1

### Synthesis of 2.(N-benzyl-N-4-methylbenzoyl)-6,7-dimethoxy-3,4-dihydro-2-naphthylamine

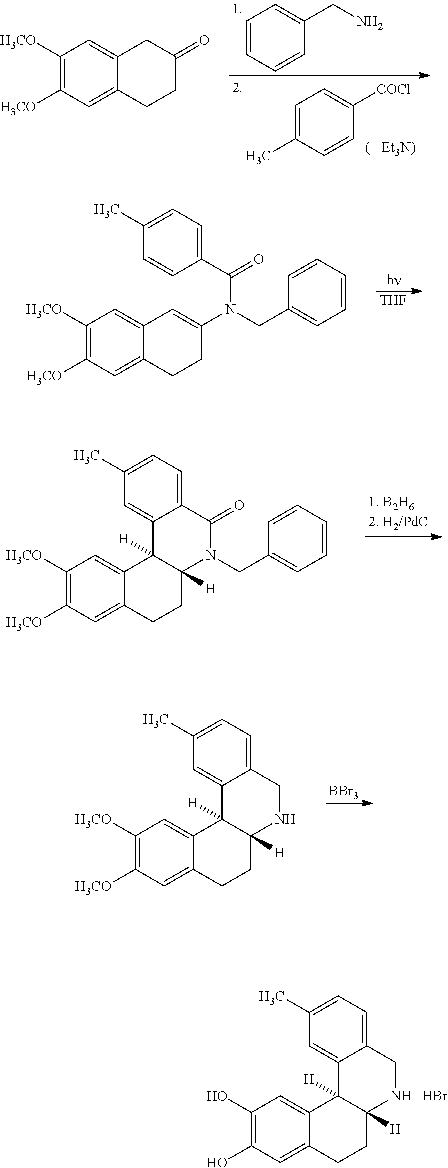

To a solution of 4.015 g (19.5 mmol) of 6,7-dimethoxy-B-tetralone in 100 ml of toluene was added 2.139 g (1.025 equiv.) of benzylamine. The reaction was heated at reflux overnight under N₂ with continuous water removal. The reaction was cooled and the solvent was removed by rotary vacuum evaporation to yield the crude N-benzyl enamine as a brown oil.

Meanwhile, the 4-methylbenzoyl chloride acylating agent was prepared by suspending 3.314 g (24.3 mmol) of p-toluic acid in 200 ml benzene. To this solution was added 2.0 equiv. (4.25 ml) of oxalyl chloride, dropwise via a pressure-equalizing dropping funnel at 0° C. DMF (2-3 drops) was added to the reaction mixture catalytically and the ice bath was removed. The progress of the reaction was monitored via infrared spectroscopy. The solvent was removed by rotary vacuum evaporation and the residual oil was pumped down under high vacuum overnight.

The crude N-benzyl enamine residue was dissolved in 100 ml of CH₂Cl₂, and to this solution was added 2.02 g (19.96 mmol) of triethylamine at 0° C. 4-methylbenzoyl chloride (3.087 g, 19.96 mmol) was dissolved in 20 ml CH₂Cl₂ and this solution was added dropwise to the cold, stirring N-benzyl enamine solution. The reaction was allowed to warm to room temperature and was left to stir under N₂ overnight. The reaction mixture was washed successively with 2×30 ml of 5% aqueous HCl, 2×30 ml of saturated sodium bicarbonate solution, saturated NaC1 solution, and was dried over MgSO₄. After filtration, the filtrate was concentrated under vacuum. Crystallization from diethyl ether gave 5.575 g (69.3%) of the enamide mp 96°-98° C. CIMS (isobutane); M+1 414; ¹H-NMR (CDCl₃); δ7.59 (d, 2, ArH), 7.46 (m, 3, ArH), 7.35 (m, 3, ArH), 7.20 (d, 2, ArH), 6.60 (s, 1, ArH), 6.45 (s, 1, ArH), 6.18 (s, 1, ArCH), 5.01 (s, 2, ArCH₂ N), 3.80 (S, 3, OCH₃), 3.78 (s, 3, OCH₃), 2.53 (t, 2, ArCH₂), 2.37 (s, 3, ArCH₃), 2.16 (t, 2, CH₂); Anal. (C₂₇H₂₇NO₃) C, H, N.

Trans-2-methyl-6-benzyl-10,11-dimethoxy-5,6,6a,7,8,12b-hexahydro-benzo[a]phenanthridine-5-one.

A solution of 4.80 g (11.62 mmol) of the 6,7-dimethoxy enamide prepared above, in 500 ml of THF, was introduced to an Ace Glass 500 ml photochemical reactor. This solution was stirred while irradiating for 2 hours with a 450 watt Hanovia medium pressure, quartz, mercury-vapor lamp seated in a water-cooled, quartz immersion well. The solution was concentrated in vacuo and crystallized from diethyl ether to provide 2.433 (50.7%) of the 10,11-dimethoxy lactam, mp 183°-195° C. CIMS (isobutane); M+1 414; ¹H-NMR (CDCl₃); δ8.13 (d, 1, ArH), 7.30 (s, 1, ArH), 7.23 (m, 6, ArH), 6.93 (s, 1, ArH), 6.63 (s, 1, ArH), 5.38 (d, 1, ArCH₂ N), 5.30 (d, 1, ArCH₂ N), 4.34 (d, 1, Ar₂ CH, J=11.4 Hz), 3.89 (s, 3, OCH₃), 3.88 (s, 3, OCH₃), 3.76 (m, 1, CHN), 2.68 (m, 2, ArCH₂), 2.37 (s, 3, ArCH₃), 2.25 (m, 1, CH₂ CN), 1.75 (m, 1, CH₂ CN); Anal. (C₂₇H₂₇NO₃) C, H, N.

Trans-2-methyl-6-benzyl-10,11-dimethoxy-5,6,6a,7,8,12b-hexahydrobenzo[a]phenanthridine hydrochloride.

A solution of 1.349 g (3.27 mmol) of the lactam prepared above, in 100 ml dry THF was cooled in an ice-salt bath and 4.0 equiv. (13.0 ml) of 1.0 molar BH₃ was added via syringe. The reaction was heated as reflux under nitrogen overnight. Methanol (10 ml) was added dropwise to the reaction mixture and reflux was continued for 1 hour. The solvent was removed by rotary vacuum evaporation. The residue was chased two times with methanol and twice with ethanol. The flask was placed under high vacuum (0.05 mm Hg) overnight. The residue was dissolved in ethanol and was carefully acidified with concentrated HCl. The violatiles were removed and the product was crystallized from ethanol to afford 1.123 g (78.9%) of the hydrochloride salt, mp 220°-223° C. CIMS (isobutane); M+1 400; ¹H-NMR (CDCl₃, free base); δ7.37 (d, 2, ArH), 7.33 (m, 2, ArH), 7.26 (m, 1, ArH), 7.22 (s, 1, ArH), 7.02 (d, 1, ArH), 6.98 (d, 1, ArH), 6.89 (s, 1, ArH), 6.72 (s, 1, ArH), 4.02 (d, 1, Ar₂ CH, J=10.81 Hz), 3.88 (s, 3, OCH₃), 3.86 (d, 1, ArCH₂ N), 3.82 (m, 1, ArCH₂ N), 3.78 (s, 3, OCH₃), 3.50 (d, 1, ArCH₂N), 3.30 (d, 1, ArCH₂ N), 2.87 (m, 1, ArCH₂), 2.82 (m, 1, CHN), 2.34 (m, 1, CH₂CN), 2.32 (s, 3, ArCH₃), 2.20 (m, 1, ArCH₂), 1.93 (m, 1, CH₂ CN); Anal. (C₂₇H₂₉NO₂C, H, N.

Trans-2-methyl-10,11-dimethoxy-5,6,6a,7,8,12b-hexahydrobenzo[a]phenanthridine hydrochloride.

A solution of 0.760 g (1.75 mmol) of the 6-benzyl hydrochloride salt prepared above in 100 ml of 95% ethanol containing 150 mg of 10% Pd/C catalyst was shaken at room temperature under 50 psig of H₂ for 8 hours. After removal of the catalyst by filtration through Celite, the solution was concentrated to dryness under vacuum and the residue was recrystallized from acetonitrile to afford 0.520 g (86.2%) of the crystalline salt, mp 238° -239° C. CIMS (isobutane); M+1 310; ¹H-NMR (DMSO, HCl salt); δ10.04 (s, 1, NH), 7.29 (d, 1, ArH), 7.16 (m, 2, ArH), 6.88 (s, 1, ArH), 6.84 (s, 1, ArH), 4.31 (s, 2, ArCH₂N), 4.23 (d, 1, Ar₂CH, J=10.8 Hz), 3.76 (s, 3, OCH₃), 3.70 (s, 3, OCH₃), 2.91 (m, 2, ArCH₂), 2.80 (m, 1, CHN), 2.49 (s, 3, ArCH₃), 2.30 (m, 1, CH₂ CN), 2.09 (m, 1, CH₂ CN); Anal. (C₂₀H₂₃NO₂) C, H, N.

Trans-2-methyl-10,11-dihydroxy-5,6,6a,7,8,12b-hexahydrobenzo[a]phenanthridine hydrochloride.

0.394 g (1.140 mmol) of the O,O-dimethyl hydrochloride salt prepared above was converted to its free base. The free base was dissolved in 35 ml of dichloromethane and the solution was cooled to −78° C., 4.0 equiv. (4.56 ml) of a 1.0 molar solution of BBr₃ was added slowly via syringe. The reaction was stirred under N₂ overnight with concomitant warming to room temperature. 7.0 ml of methanol was added to the reaction mixture and the solvent was removed by rotary vacuum evaporation. The flask was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water. The filtrate was extracted several times with dichloromethane and the organic extacts were dried, filtered and concentrated. The filter cake and the organic residue were combined, dissolved in ethanol and carefully acidified with concentrated HCl. After removal of the volatiles, the HCl salt was crystallized as a solvate from methanol in a yield of 0.185 g (51%), mp (decomposes @ 190° C.). CIMS (isobutane); M+1 282; ¹H-NMR (DMSO, HCl salt); δ9.52 (s, 1, NH), 8.87 (d, 2, OH), 7.27 (d, 1, ArH), 7.20 (s, 1, ArH), 7.15 (d, 1, ArH), 6.72 (s, 1, ArH), 6.60 (s, 1, ArH), 4.32 (s, 2, ArCH₂N), 4.10 (d, 1, ArCH₂CH, J=11.26 Hz), 2.90 (m, 1, CHN), 2.70 (m, 2, ArCH₂), 2.32 (s, 3, ArCH₃), 2.13 (m, 1, CH₂CN), 1.88 (m, 1, CH₂CN); Anal. (C₁₈H₁₉NO₂) C, H, N.

Using similar techniques, compounds shown below in Table 1 can be prepared:

TABLE 1

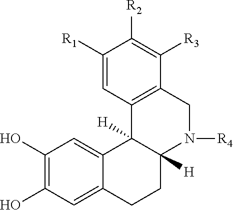

| R₁ | R₂ | R₃ | R₄ |
|---|---|---|---|
| CH₃ | H | H | H |
| CH₃ | H | H | CH₃ |
| CH₃ | H | H | C₃H₇ |
| CH₃ | H | CH₃ | C₃H₇ |
| CH₃ | CH₃ | H | H |
| Cl | H | H | H |
| Cl | H | H | C₃H₇ |
| Br | H | H | H |
| Br | H | H | C₃H₇ |
| F | H | H | H |
| F | H | H | C₃H₇ |
| C₂H₅ | H | H | H |
| C₂H₅ | H | H | C₃H₇ |
| C₃H₇ | H | H | H |
| C₃H₇ | H | H | C₃H₇ |

### EXAMPLE 2

### Synthesis of Compound 1 (2-methyldihydrexidine)

Synthesis of 6,7-dimethoxy-2-tetralone:

The cyclic ketone, 6,7-dimethoxy-2-tetralone is a key starting material for the synthesis of the full dopamine agonist dihydrexidine and its derivatives. It was prepared cost-effectively by starting from the readily available 6,7-dimethoxy-1-tetralone. Sodium borohydride reduction gave the 1,2,3,4-tetrahydro-6,7-dimethoxy-1-naphthalenol, which was then converted to its olefin, followed by oxidation to afford an epoxide. This epoxide, on treatment with BF₃-ether furnished 6,7-dimethoxy-2-tetralone (Scheme 2).

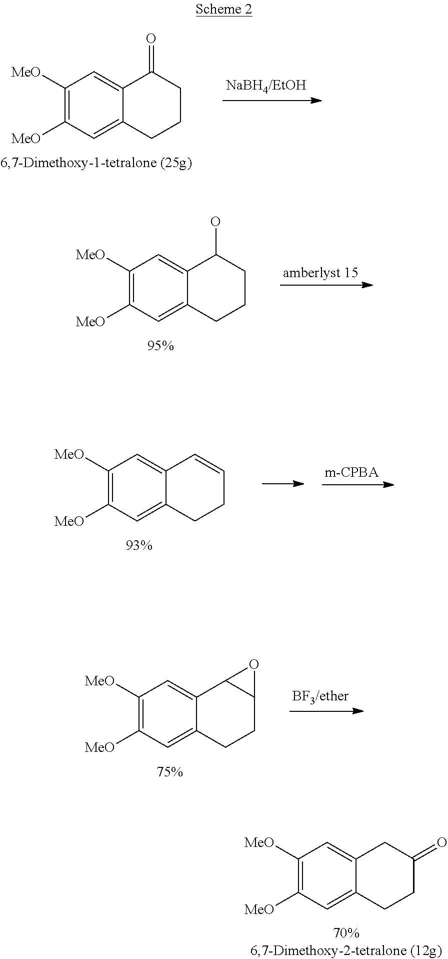

Then, 2-methyldihydrexidine (7) was synthesized by previously described methods (Knoerzer et al., 1995) that have been modified slightly (Scheme 3).

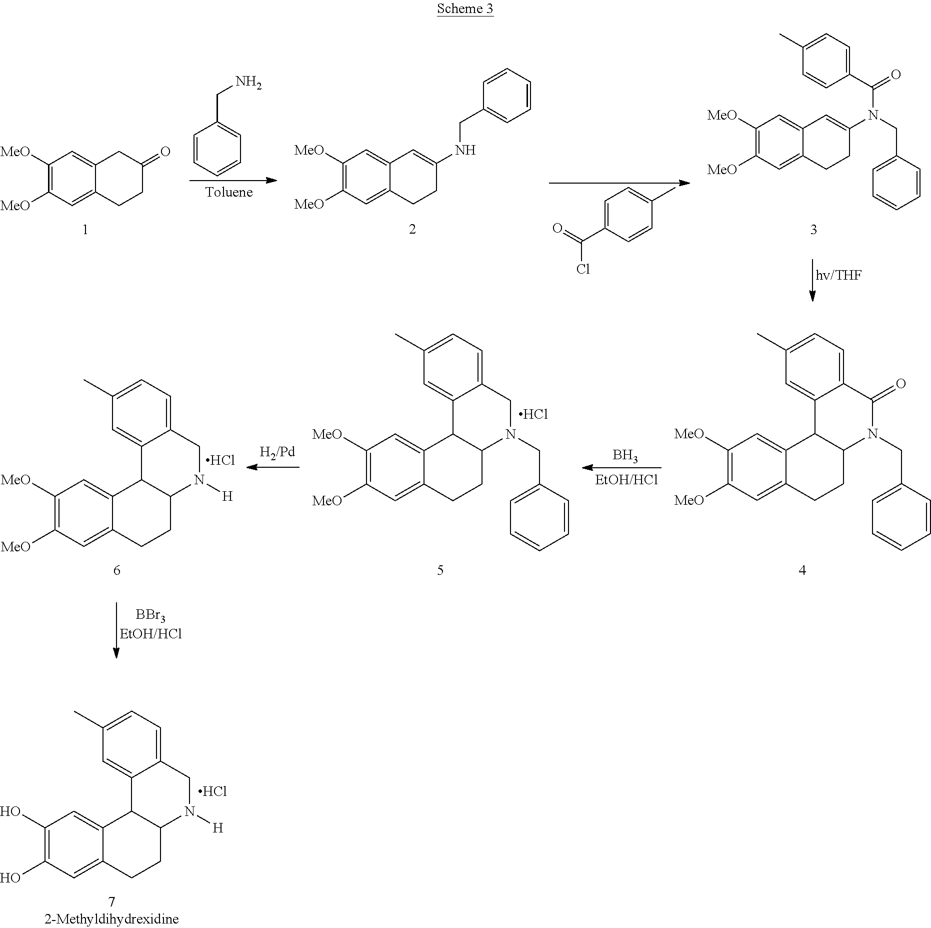

Briefly, a solution of 6,7-dimethoxy-2-tetralone (1) in toluene was refluxed with benzylamine with continuous water removal via a Dean-Stark apparatus to yield the crude N-benzyl enamine (2). The crude enamine was stirred overnight with p-toluoyl chloride and triethylamine in dichloromethane, letting the temperature to rise from 0° C. to ambient temperature overnight. The enamide (3) thus formed was purified by silica gel column chromatography and subjected to photochemical reaction in THF to yield cyclized product 4. The ketone (4) was reduced by BH₃-THF solution to give 5, which on de-benzylation by 10% Pd-C catalytic hydrogenation resulted in the formation of O,O-dimethyl ether hydrochloride salt 6. The compound 6 was finally converted into free base and treated with BBr₃ to obtain 2-methyldihydrexidine (7). The free base was converted to hydrochloride salt again by EtOH/HCl. The structure of compound 7 was confirmed by NMR and MS data; mp 208-210° C.; ′H NMR (DMSO) δ69.53 (s, 1, NH), 9.79 (s, 2, OH), 7.27 (d, 1, ArH, J=8 Hz), 7.22 (s, 1, ArH), 7.14 (d, 1, ArH, J=8 Hz), 6.73 (s, 1, ArH), 6.62 (s, 1, ArH), 4.31 (S, 2, ArCH₂N), 4.11 (d, 1, Ar₂CH, J=11 Hz), 2.90 (m, 1, CHN), 2.70 (m, 2, ArCH₂), 2.32 (s, 3, ArCH₃), 2.16 (m, 1, CHCN), 1.92 (m, 1,CHCN). ESI-MS 282 [M+H]⁺.

Step 1: N-(4′-Methylbenzoyl)-N-benzyl-6,7-dimethoxy-3,4-dihydro-2-naphthylamine (3)

A solution of 6,7-dimethoxy-2-tetralone 1 (5.0 g, 24.25 mmol) in 150 mL of toluene was stirred at room temperature under a nitrogen atmosphere while 2.85 g (26.66 mmol) of benzylamine is added to the solution. The reaction mixture was heated at reflux overnight under nitrogen with continuous water removal via a Dean-Stark apparatus. The reaction mixture was cooled to room temperature, and the solvent is removed in vacuo to yield the crude N-benzyl enamine (2) as a brown oil.

The crude enamine was dissolved in 150 mL of dichloromethane, and the solution was cooled to 0° C. in an ice bath. Triethylamine (1.1 equiv) was added to the solution with stirring. The p-toluoyl chloride (4.03 g, 26 mmol) was dissolved in 20 mL of dichloromethane, and this solution was added dropwise to the cold, stirring enamine solution. After complete addition, the ice bath was removed, and the reaction mixture was left to stir overnight at room temperature under a nitrogen atmosphere. The reaction mixture was washed with 2×50 mL of 5% HCl, 2×50 mL of saturated NaHCO₃ solution, and brine. The organic phase was dried with MgSO₄, filtered, and concentrated. The crude product was passed over a silica gel flash column, eluting with 5% ether in dichloromethane. The collected fractions containing the product were combined and concentrated. The product was crystallized from Et₂O, mp 89-90° C., (Yield 70%).

Step 2: (+)-trans-2-Methyl-6-benzyl-10,11-dimethoxy-5,6,6a,7,8,12b hexahydrobenzo[a]phenanthridin-5-one (4)

A solution of enamide 3 (3.0 g, 7.26 mmol) was prepared in 1 L of THF. This solution was placed into an Ace glass 1 L photochemical reactor. The solution was stirred while irradiating with a 450 W Hanovia medium pressure, quartz, mercury-vapor lamp seated in a cold tap water-cooled, quartz immersion well. When TLC analysis had indicated the complete disappearance of the starting material (˜3 h), the solution was concentrated via rotary evaporation. The product was purified by elution through a silica gel flash column with 5% ether in dichloromethane. The appropriate fractions were combined, and the product was crystallized from diethyl ether, mp 183-185° C., (Yield 54%).

Step 3: (+)-trans-2-Methyl-6-benzyl-10,11-dimethoxy-5,6,6a,7,8,12b-hexahydrobenzo[a]phenantridine hydrochloride (5)

A solution of 4 (4.0 g, 10.02 mmol) in 200 mL of dry THF is cooled in an ice-salt bath, and 4-5 equiv of a 1.0 M solution of BH₃ in THF was added via syringe. The reaction mixture was heated at reflux until all of the starting material had been consumed (TLC, 5% Et₂O/CH₂Cl₂). Methanol (70 mL) was then added cautiously to the reaction mixture, and the mixture was heated at reflux for an additional 4 h. The reaction mixture was cooled to room temperature, and the solvent was removed in vacuo. Methanol (50 mL) was added to the flask and then removed on the rotary evaporator; this procedure was repeated. Likewise, two 50 mL portions of ethanol were added and removed in the same manner. The reaction flask was then left under high vacuum overnight. The residue was suspended in absolute EtOH and carefully acidified with concentrated HCl. The volatiles were removed, and the product was crystallized from ethanol, mp 220-223° C., (Yield 80%).

Step 4: (+)-trans-2-Methyl-10,11-dimethoxy-5,6,6a,7,8,12b hexahydrobenzo[a]phenantridine hydrochloride (6)

A solution of 6-N-benzyl hydrochloride salt 5 (2.8 g, 7.01 mmol) in 100 mL of methanol containing 0.60 g of 10% Pd-C catalyst is shaken at room temperature under 50 psi of H₂ overnight. The catalyst is removed by filtration through a pad of celite. The solution is concentrated to dryness on a rotary evaporator, and the product is crystallized from acetonitrile, mp 238-240° C., (Yield 88%).

Step 5: (+)-trans-2-Methyl-10,11-dihydroxy-5,6,6a,7,8,12b hexahydrobenzo[a]phenantridine hydrochloride (7)

The O,O-dimethyl ether hydrochloride salt 6 (1.8 g, 5.82 mmol) was converted to their free bases in H₂O with a saturated bicarbonate solution. The aqueous solution was extracted with 3×30 mL of dichloromethane. The organic fractions were dried over MgSO₄, filtered, and concentrated in vacuo. The free base was dissolved in 35 mL of dichloromethane, and the solution was cooled to −78° C. A 1.0 M solution of BBr₃ in dichloromethane (30 mmol, 4-5 equiv) was added slowly to the reaction mixture via syringe. The cooling bath was removed, and the reaction mixture was left to stir under a nitrogen atmosphere overnight, while warming to ambient temperature. Methanol (50 mL) was then added cautiously to the reaction mixture over 20 min. The solvent was removed by rotary evaporation, and the flask was left under high vacuum overnight. The residue was dissolved in water and carefully neutralized to its free base with a saturated bicarbonate solution while cooling in an ice bath. The free base was filtered and washed with cold water. The precipitated free base was dissolved in absolute ethanol, and carefully acidified with concentrated HCl. After removal of the volatiles, the hydrochloride salt was crystallized from MeOH/EtOAc, mp 208-210° C., (Yield 52%).

### EXAMPLE 3

### Binding Affinity of Compound 1

The affinity of Compound 1, relative to dihydrexidine, chlorpromazine, and SCH23390, for D₁ and D₂ binding sites was assayed using rat brain striatal homogenates having D₁ and D₂ binding sites labeled with ³H-SCH 23390 and ³H-spiperone, respectively. The data are shown in Table 2.

TABLE 2
Dopamine receptor binding affinities of Compound 1^{A}

| | Rat striatal | | Cloned human receptors | | | | |
|---|---|---|---|---|---|---|---|
| | homogenates | | | | D₃ | D₄ | |
| Drug | D₁ | D₂ | D₁ (C-6) | D_{2L} (C-6) | (CHO) | (CHO) | D₅ (HEK) |
| SCH23390 | 0.4 | -nt- | 0.4 | -nt- | -nt- | -nt- | 1.2 |
| chlorpromazine | -nt- | 2.0 | -nt- | 0.74 | 0.86 | 20 | -nt- |
| dihydrexidine | 6.5 | 50 | 2.2 | 180 | 15 | 14 | 14 |
| Compound 1 | 9.0 | 325 | 8.0 | 525 | 95 | 325 | 7.0 |

^{A}All values are K_{0.5} in nM concentrations
nt = not tested

As is well known, the affinity and potency of agonists for members of the GPCR superfamily is affected markedly by receptor expression and by the cellular milieu in which the receptor is expressed. For this reason, rat striatal membranes were used as a standard of comparison. In all of these preparations, Compound 1 was a high affinity full D₁ agonist with a K_{0.5} slightly higher than dihydrexidine. In addition, Compound 1 was somewhat more selective for the D₂ family of receptors than was dihydrexidine (Table 2). It is believed that this compound is D₂ functionally selective, and therefore may decrease nausea that is common with compounds that activate D₂ receptors.

Affinity of Compound 1 at Other Receptors:

Compound 1 was tested for its affinity to a host of other potential receptor targets. The strategy used first examined the effects of a 10 μM concentration of Compound 1, and then did full dose-response studies for any receptor in which there was >50% inhibition of binding. For non-dopamine receptors, Compound 1 had little affinity for most receptors that might have been predicted to be engaged by its structural features. This includes various monoamine transporters (DAT, NERT; SERT); many serotonin receptors (5-HT_{1D}; r5-HT_{2A}; 5-HT_{2B}; 5-HT₃; 5-HT₆; adrenergic receptors (α_{1A}; α_{1B}; α_{2A}; α_{2B}; α_{2C}; rβ₁; rβ₂); hH1; muscarinic receptors (M1; M2; M3; M4; M5); opioids (μ; κ); and a variety of miscellaneous receptors (e.g., EP3; EP4; rPCP; rBZP; etc.). From what is known about the properties of these receptors, there are no obvious concerns about the pharmacological profile of Compound 1.

### EXAMPLE 3

### Dopamine D₁ Agonist Activity of Compound 1

The activity of Compound 1 as a dopamine agonist was measured. Studies were done in cloned hDl receptors expressed in Ltk cells. Compound 1 was a full agonist of similar intrinsic activity and potency when compared to dihydrexidine. The data is shown in FIG. 2.

The agonist effects of Compound 1 were completely blocked by the D₁ antagonist SCH23390 (0.3 mg/kg). This data demonstrates the D₁ activity of Compound 1. However, the rotation caused by Compound 1 was not blocked by the D₂ antagonist haloperidol (0.1 mg/kg), which demonstrates the D₁ selectivity over D₂.

Behavioral Effects of Compound 1 in the Rat Model of Parkinson's Disease

The activity of Compound 1 in a unilateral 6-OHDA rat model was measured, and compared to the activity of dihydrexidine. The results are shown in FIG. 1. As shown in FIG. 1 (top chart), a single subcutaneous injection of 1 mg/kg Compound 1 produced activity lasting for nearly ten hours (N=8). As shown in FIG. 1 (bottom chart), dihydrexidine is much shorter acting, with no activity present after three hours (N=4, consistent with earlier dihydrexidine studies.).

A critical characteristic of a drug for use in Parkinson's disease is that it does not have an overly short duration of action as do dihydrexidine and almost all other full D₁ agonists. One of the reasons for the short duration of action is believed to be the presence of the catechol moiety that is, so far, essential for full agonist actions (Mailman et al., 2001; Mailman and Huang, 2007; Mottola et al., 1996). This makes such compounds metabolically labile by phase 2 drug metabolism. Thus, Compound 1 was selected form a broad genus of hexahydrobenzophenanthridines when it was unexpectedly found to have much better pharmacokinetic properties. This is illustrated in FIGS. 3A and 3B, with comparison data to dihydrexidine. The duration of action is expected to correlate with peripheral drug levels.

Methods

Animals

In some studies, adult male Sprague Dawley rats (200-250 g) were obtained from Charles River Breeding Laboratories (Raleigh, N.C.) or Harlan Laboratories (Indianapolis, Ind.). Rats were killed by decapitation, and the whole brains removed and chilled briefly in ice-cold saline. Brains were sliced with the aid of a dissecting block, and central striata were then dissected from two coronal sections containing the majority of this region. Tissue was frozen immediately on dry ice and stored at −70° C. until the day of the assay. In other studies, Sprague-Dawley rats weighing between ca . 175 g received unilateral injections of 6-OHDA. They were pretreated with 25 mg/kg desipramine, anesthetized with sodium pentobarbital and placed in a stereotaxic apparatus. Thirty gauge needles were lowered into the right aspects of the substantia nigra at Interaural zero +3.2 mm; lateral −2.0 mm; and dorsoventral −7.7 mm. Four μl of 0.1% ascorbic acid in sterile saline containing 8 μg of 6-OHDA were infused in each side over a period of 20 minutes using 10 μl Hamilton syringes and a Sage syringe pump. The needles were left in place two minutes after infusion to minimize withdrawal of the 6-OHDA solution with the needles. The small burr holes in the skull were plugged with bone wax, and the wound sutured. Post-operative care of these rats included observation, sweet food supplements, and gastric intubation during the aphagic period following lesion. Fourteen or more days after the lesion, rats were challenged with 1 mg/kg apomorphine, and rotations measured. Rats that failed to meet criterion (100 rotations in an hour) were not used further. The rats were then allowed to “wash-out” for a week between drug trials.

Cell Cultures

C-6 glioma cells expressing the rhesus macaque D₁ A receptor, (C-6-mD_{1A}; Machida et al., 1992) were grown in DMEM-H medium containing 4,500 mg/L glucose, L-glutamine, 5% fetal bovine serum and 600 ng/mL G418 or 2 μg/mL puromycin. Cells were maintained in a humidified incubator at 37° C. with 5% CO₂.

Membrane Preparation

Cells were grown in 75 cm² flasks until confluent. The cells were rinsed and lysed with 10 mL of ice cold hypoosmotic buffer (HOB) (5 mM Hepes, 2.5 mM MgCl₂, 1 mM EDTA; pH 7.4) for 10 minutes at 4° C. Cells were then scraped from the flasks using a sterile cell scraper from Baxter (McGaw Park, Ill.). Flasks received a final rinse with 5 mL of HOB. The final volume of the cell suspension recovered from each flask was ca. 14 mL. Scraped membranes from several flasks were then combined. The combined cell suspension was homogenized (10 strokes), 14 mL at a time, using a 15 mL Wheaton Teflon-glass homogenizer. The cell homogenates were combined and spun at 43,000×g (Sorvall RC-5B/SS-34, DuPont, Wilmington, Del.) at 4° C. for 20 min. The supernatant was removed, and the pellet was resuspended (10 strokes) in 1 mL of ice cold HOB for each original flask of cells homogenized. This homogenate was then spun again at 43,000×g at 4° C. for 20 min. The supernatant was removed and the final pellet was resuspended (10 strokes) in ice cold storage buffer (50 mM Hepes, 6 mM MgCl₂, 1 mM EDTA; pH 7.4) to yield a final concentration of ca. 2.0 mg of protein/mL. Aliquots of the final homogenate were stored in microcentrifuge tubes at −80° C. Prior to their use for adenylate cyclase assays, protein levels for each membrane preparation were quantified using the BCA protein assay reagent (Pierce, Rockford, Ill.) adapted for use with a microplate reader (Molecular Devices; Menlo Park, Calif.).

Dopamine Receptor Binding Assays

Frozen rat striata were homogenized by seven manual strokes in a Wheaton Teflon-glass homogenizer in 8 mL ice cold 50 mM HEPES buffer with 4.0 mM MgCl₂ (pH 7.4). Tissue was centrifuged at 27,000×g for 10 min, the supernatant was discarded, and the pellet was homogenized (five strokes) and resuspended in ice cold buffer and centrifuged again. The final pellet was suspended at a concentration of 2.0 mg wet weight/mL. The amount of tissue added to each assay tube was 1.0 mg, in a final assay volume of 1.0 mL. D₁ receptors were labeled with [³H]SCH23390 (0.30 nM); D₂ receptors were labeled with [³H]spiperone (0.07 nM); unlabeled ketanserin (50 nM) was added to mask binding to 5-HT₂-type receptors. Total binding was defined as radioligand bound in the absence of any competing drug. Nonspecific binding was estimated by adding unlabeled SCH23390 (1 μM) or unlabeled chlorpromazine (1 μM) for D₁ and D₂ receptor binding assays, respectively. As an internal standard, a competition curve with six concentrations of unlabeled SCH23390 (D₁ binding) or chlorpromazine (D₂ binding) was included in each assay. Triplicate determinations were made for each drug concentration. Assay tubes were incubated at 37° C. for 15 minutes, and binding was terminated by filtering with ice cold buffer on a Skatron 12 well cell harvester (Skatron, Inc., Sterling, Va.) using glass fiber filter mats (Skatron no. 7034). Filters were allowed to dry, and 1.0 mL of Optiphase HI-SAF II scintillation fluid was added. Radioactivity was determined on an LKB Wallac 1219 RackBeta liquid scintillation counter (Wallac, Gaithersburg, Md.). Tissue protein levels were estimated using the BCA protein assay reagent (Pierce, Rockford, Ill.).

Data Analysis for Radioreceptor Assays

Binding data from each assay were analyzed separately. Data were normalized by expressing the average dpm at each competitor concentration as a percentage of total binding. These data were then subjected to nonlinear regression analysis using the algorithm for sigmoid curves in the curve-fitting program InPlot (Graphpad Inc.; San Francisco, Calif.) to generate Ko. values and a Hill coefficient (n_{H}) for each curve. Analysis of the residuals indicated an excellent fit; r values were above 0.99 for all curves in the present experiments.

Adenylate Cyclase Assay in C-6mD_{1A} Cells

Frozen membranes were thawed and added to assay tubes (10 μg protein/tube) containing a prepared reaction mixture [100 mM Hepes, (pH 7.4), 100 mM NaCl, 4 mM MgCl₂, 2 mM EDTA, 500 μM isobutyl methylxanthine (IBMX), 0.01% ascorbic acid, 10 μM pargyline, 2 mM ATP, 5 μM GTP, 20 mM phosphocreatine, 5 units of creatine phosphokinase (CPK), 1 μM propranolol] and selected drugs. The final reaction volume was 100 μL.

Basal cAMP activity was determined by incubation of tissue in the reaction mixture with no drug added. Tubes were assayed in duplicate and, after a 15 min incubation at 30° C., the reaction was stopped by the addition of 500 μL of 0.1 N HCl. Tubes were vortexed briefly, and then spun in a BHG HermLe Z 230 M microcentrifuge for five min at 15,000×g to precipitate particulates.

Radioimmunoassay (RIA) of cAMP

The concentration of cAMP in each sample was determined with an RIA of acetylated cAMP, modified from that previously described. Iodination of cAMP was performed using a now-published procedure (Brown et al., 2009). Assay buffer was 50 mM sodium acetate buffer with 0.1% sodium azide (pH 4.75). Standard curves of cAMP were prepared in buffer at concentrations of 2 to 500 fmol/assay tube. To improve assay sensitivity, all samples and standards were acetylated with 10 μl of a 2:1 solution of triethylamine:acetic anhydride. Samples were assayed in duplicate. Each assay tube (total volume 300 μL) contained 25 μL of each sample, 75 μL of buffer, 100 μL of primary antibody (sheep, anti-cAMP, 1:100,000 dilution with 1% BSA in buffer) and 100 μL of [¹²⁵I]-cAMP (50,000 dpm/100 μL of buffer). Tubes were vortexed and stored at 4° C. overnight (approx. 18 h). Antibody-bound radioactivity was separated by the addition of 25 μL of BioMag rabbit, anti-goat IgG (Advanced Magnetics, Cambridge Mass.), followed by vortexing and incubation at 4° C. for 1 h. To these samples 1 mL of 12% polyethylene glycol/50 mM sodium acetate buffer (pH 6.75) was added and tubes were centrifuged at 1700×g for 10 min. Supernatants were aspirated and radioactivity in the pellet was determined using an LKB Wallac gamma counter (Gaithersburg, Md.).

Data Analysis for Adenylate Cyclase Studies

Data for each sample were expressed initially as pmol/mg/min cAMP. Baseline values of cAMP were subtracted from the total amount of cAMP produced in each drug condition. Data for each drug were expressed relative to the stimulation produced by 100 μM DA.

### REFERENCES

The following references were cited herein, and the contents of each of these is hereby incorporated by reference in its entirety.

Arnsten A F, Cai J X, Murphy B L, Goldman-Rakic P S. 1994. Dopamine D1 receptor mechanisms in the cognitive performance of young adult and aged monkeys. Psychopharmacology (Berl) 116:143-151.

Asano Y, Yamashita M, Nagai K, Kuriyama M, Yamada K, Tomioka K. 2001. The first asymmetric synthesis of a dopamine D1 agonist, dihydrexidine, employing asymmetric conjugate addition technology. Tetrahedron Letters 42:8493-8495.

Bach M E, Barad M, Son H, Zhuo M, Lu Y F, Shih R, Mansuy I, Hawkins R D, Kandel E R. 1999. Age-related defects in spatial memory are correlated with defects in the late phase of hippocampal long-term potentiation in vitro and are attenuated by drugs that enhance the cAMP signaling pathway. Proc Natl Acad Sci USA 96:5280-5285.

Brown J T, Kant A, Mailman R B. 2009. Rapid, semi-automated, and inexpensive radioimmunoassay of cAMP: Application in GPCR-mediated adenylate cyclase assays. J Neurosci Methods 177:261-266.

Brozoski T J, Brown R M, Rosvold H E, Goldman P S. 1979. Cognitive deficit caused by regional depletion of dopamine in prefrontal cortex of rhesus monkey. Science 205:929-932.

Cai J X, Arnsten A F. 1997. Dose-dependent effects of the dopamine D1 receptor agonists A77636 or SKF81297 on spatial working memory in aged monkeys. J Pharmacol Exp Ther 283:183-189.

Caine S B, Koob G F, Parsons L H, Everitt B J, Schwartz J C, Sokoloff P. 1997. D3 receptor test in vitro predicts decreased cocaine self-administration in rats. NeuroReport 8:2373-2377.

Carlsson A. 1959. The occurrence, distribution and physiological role of catecholamines in the nervous system. Pharmacol Rev 11:493.

Carlsson A, Lindqvist M, Magnusson T, WALDECK B. 1958. On the presence of 3-hydroxytyramine in brain. Science 127:471.

Castner S A, Williams G V, Goldman-Rakic PS. 2000. Reversal of antipsychotic-induced working memory deficits by short-term dopamine D1 receptor stimulation. Science 287:2020-2022.

Cederbaum J M, Schleifer L S. 1990. Drugs for Parkinson's Disease, spasticity, and acute muscle spasms. In: Gilman AG, Rall T, Nies A, Taylor P, editors. Goodman & Gilman's The Pharmacological Basis of Therapeutics. Pergamon Press. pp. 463-484.

Cheng Y, Prusoff W H. 1973. Relationship between the inhibition constant (K₁) and the concentration of inhibitor which causes 50 per cent inhibition (ISO) of an enzymatic reaction. Biochem Pharmacol 22:3099-3108.

Cotzias G C, Papavasiliou P S, Gellene R. 1969. Modification of Parkinsonism—chronic treatment with L-dopa. New England Journal of Medicine 280:337-345.

Ehringer H, Hornykiewicz O. 1960. Verteilung vn Noradrenalin und Dopamin (3-hydroxytyramine) in Gehirn des Menshen und ihr Verhalten bei Evkrankungen des extrapyramidalen systems. Klin. Wschr. Klin Wochenschr 38:1236-1239.

Frey U, Matthies H, Reymann K G, Matthies H. 1991. The effect of dopaminergic D1 receptor blockade during tetanization on the expression of long-term potentiation in the rat CA1 region in vitro. Neurosci Lett 129:111-114.

Hansch C, Hoekman D, Leo A, Zhang L, Li P. 1995. The expanding role of quantitative structure-activity relationships (QSAR) in toxicology. Toxicol Lett 79:45-53.

Hansch C, Steward A R, Anderson S M, Bentley D L. 1968. Parabolic dependence of drug action upon lipophilic character as revealed by a study of hypnotics. J Med Chem 11:1-11.

Haughn Z. Levodopa: life begins at 40? Practical Neurology 6[4], pp. 24-28. 2007. Plymouth Meeting Pa., Avondale Medical Publications.

Hornykiewicz O. 1963. Die Topische Lokalisation und das verhalten von noradrenalin und dopamine (3-Hydroxytyramin) in der substantia nigra des normalen und parkinsonkranken menschen. Wien Klin Wochenschr 75:309-312.

Hoyer D, Boddeke H W. 1993. Partial agonists, full agonists, antagonists: dilemmas of definition. Trends Pharmacol Sci 14:270-275.

Huang X, Lawler C P, Lewis M M, Nichols D E, Mailman R B. 2001. D1 dopamine receptors. Int Rev Neurobiol 48:65-139.

Huang Y Y, Kandel E R. 1995. D1/D5 receptor agonists induce a protein synthesis-dependent late potentiation in the CA1 region of the hippocampus. Proc Natl Acad Sci USA 92:2446-2450.

Jenner P, Demirdemar R. 1997. Dopamine receptor sub-types: from basic sciences to clinical applications. Burke, Va.:IOS Press.

Kilts J D, Connery H S, Arrington E G, Lewis M M, Lawler C P, Oxford G S, O'Malley K L, Todd R D, Blake B L, Nichols D E, Mailman R B. 2002. Functional selectivity of dopamine receptor agonists. II. Actions of dihydrexidine in D2L receptor-transfected MN9D cells and pituitary lactotrophs. J Pharmacol Exp Ther 301:1179-1189.

Knoerzer T A, Watts V J, Nichols D E, Mailman R B. 1995. Synthesis and biological evaluation of a series of substituted benzo[a]phenanthridines as agonists at D1 and D2 dopamine receptors. J Med Chem 38:3062-3070.

Lidow M S, Goldman-Rakic P S, Gallager D W, Rakic P. 1991. Distribution of dopaminergic receptors in the primate cerebral cortex: quantitative autoradiographic analysis using [3H]raclopride, [3H]spiperone and [3H]SCH23390. Neuroscience 40:657-671.

Mailman R, Huang X, Nichols D E. 2001. Parkinson's disease and D1 dopamine receptors. Curr Opin Investig Drugs 2:1582-1591.

Mailman R B. 2007. GPCR functional selectivity has therapeutic impact. Trends Pharmacol Sci 28:390-396.

Mailman R B, Huang X. 2007. Dopamine Receptor Pharmacology. In: Koller W, Melamed E, editors. Handbook of Clinical Neurology (3rd Series) Parkinson's Disease and Related Disorders. Amsterdam:Elsevier. pp. 77-105.

Mailman R B, Nichols D E. 1998. Dopamine D1 receptor agonists as antiparkinson drugs. Trends Pharmacol Sci 19:255-256.

Matthies H, Becker A, Schroeder H, Kraus J, Hollt V, Krug M. 1997. Dopamine D₁-deficient mutant mice do not express the late phase of hippocampal long-term potentiation. NeuroReport 8:3533-3535.

Mottola D M, Kilts J D, Lewis M M, Connery H S, Walker Q D, Jones S R, Booth R G, Hyslop D K, Piercey M, Wightman R M, Lawler C P, Nichols D E, Mailman R B. 2002. Functional selectivity of dopamine receptor agonists. I. Selective activation of postsynaptic dopamine D2 receptors linked to adenylate cyclase. J Pharmacol Exp Ther 301:1166-1178.

Murphy B L, Arnsten A F, Goldman-Rakic P S, Roth R H. 1996. Increased dopamine turnover in the prefrontal cortex impairs spatial working memory performance in rats and monkeys. Proc Natl Acad Sci USA 93:1325-1329.

Negash K, Nichols D E. 2001. A new aproach for the synthesis of (+/−)-trans-10,11-dihydroxy-5,6,6a,7,8,12b-Hexahydrobenzo[a]phenanthridine (Dihydrexidine). Tett Lett 37:6971-6972.

Neve K A, Neve R L. 1997. The dopamine receptors. Totowa, N.J.:Humana Press.

Neve K A, Seamans J K, Trantham-Davidson H. 2004. Dopamine receptor signaling. J Recept Signal Transduct Res 24:165-205.

Parkinson J. 1817. An Essay on the Shaking palsy. London:Sherwood, Neely and Jones.

Qandil A M, Miller D W, Nichols D E. 1999. A practical and cost-effective synthesis of 6,7-dimethoxy-2-tetralone. Synthesis 1999:2033-2035.

Sawaguchi T, Goldman-Rakic P S. 1991. D1 dopamine receptors in prefrontal cortex: involvement in working memory. Science 251:947-950.

Sawaguchi T, Goldman-Rakic P S. 1994. The role of D₁-dopamine receptor in working memory: local injections of dopamine antagonists into the prefrontal cortex of rhesus monkeys performing an oculomotor delayed-response task. J Neurophysiol 71:515-528.

Schneider J S, Sun Z Q, Roeltgen D P. 1994. Effects of dihydrexidine, a full dopamine D-1 receptor agonist, on delayed response performance in chronic low dose MPTP-treated monkeys. Brain Res 663:140-144.

Sealfon S C, Olanow C W. 2000. Dopamine receptors: from structure to behavior. Trends Neurosci 23:S34-S40.

Self D W, Barnhart W J, Lehman D A, Nestler E J. 1996. Opposite modulation of cocaine-seeking behavior by D1- and D2-like dopamine receptor agonists. Science 271:1586-1589.

Simon H. 1981. Dopaminergic A10 Neurons and Frontal System. Journal de Physiologie 77:81-95.

Taylor J R, Lawrence M S, Redmond D E, Jr., Elsworth J D, Roth R H, Nichols D E, Mailman R B. 1991. Dihydrexidine, a full dopamine D1 agonist, reduces MPTP-induced parkinsonism in monkeys. Eur J Pharmacol 199:389-391.

Ungerstedt U, Arbuthnott G W. 1970. Quantitative recording of rotational behavior in rats after 6-hydroxy-dopamine lesions of the nigrostriatal dopamine system. Brain Res 24:485-493.

Urban J D, Clarke W P, von Zastrow M, Nichols D E, Kobilka B, Weinstein H, Javitch J A, Roth B L, Christopoulos A, Sexton P M, Miller K J, Spedding M, Mailman R B. 2007. Functional selectivity and classical concepts of quantitative pharmacology. J Pharmacol Exp Ther 320:1-13.

Weed M R, Paul I A, Dwoskin L P, Moore S E, Woolverton W L. 1997. The relationship between reinforcing effects and in vitro effects of D1 agonists in monkeys. J Pharmacol Exp Ther 283:29-38.

Weed M R, Vanover K E, Woolverton W L. 1993. Reinforcing effect of the D1 dopamine agonist SKF 81297 in rhesus monkeys. Psychopharmacology (Berl) 113:51-52.

Weed M R, Woolverton W L. 1995. The reinforcing effects of dopamine D1 receptor agonists in rhesus monkeys. J Pharmacol Exp Ther 275:1367-1374.

Williams G V, Castner S A. 2006. Under the curve: critical issues for elucidating D1 receptor function in working memory. Neuroscience 139:263-276.

Williams G V, Goldman-Rakic P S. 1995. Modulation of memory fields by dopamine D1 receptors in prefrontal cortex. Nature 376:572-575.

Zahrt J, Taylor J R, Mathew R G, Arnsten A F. 1997. Supranormal stimulation of D1 dopamine receptors in the rodent prefrontal cortex impairs spatial working memory performance. J Neurosci 17:8528-8535.

Whereas the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

1. A compound of the formula 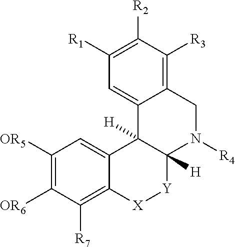
R₁ is selected from the group consisting of C₁₋₃ alkyl, C₂₋₃ alkenyl or C₂₋₃ alkynyl, alkanoyl moieties having 1 to 3 carbon atoms, halo-, and
R₂ is H or C₁₋₄ alkyl;
R₃ is H, C₁₋₇ alkyl, C₃₋₇ cycloalkyl, C₃₋₆ alkenyl, C₃₋₆ alkynyl, C₁₋₇ alkanoyl, arylalkyl, arylalkanoyl having 1 to 3 carbon atoms in the alkyl portion of the moiety, wherein the aryl ring can be substituted by fluorine, chlorine or bromine atoms;
R₄ is selected from the group consisting of H, C₁₋₃ alkyl, C₂₋₃ alkenyl, and C₂₋₃ alkynyl;
R₅ and R₆ are, independently, selected from the group consisting of H, hydroxyl protecting groups;
X and Y are, independently, C(R₈)₂, oxygen, sulfur, or NR₇, where R₇ is selected from the group consisting of H, amine protecting groups, C₁₋₇ alkyl, C₃₋₇ cycloalkyl, alkylaryl, and arylalkyl and R₈ is selected from the group consisting of H, C₁₋₇ alkyl, C₃₋₇ cycloalkyl, alkylaryl, and arylalkyl, with the proviso that at least one of X or Y must be C(R₈)₂, and wherein at least one of X and Y is oxygen, sulfur, or NR₇, or a pharmaceutically acceptable salt thereof.

2. The compound of claim 1 wherein R₁ is methyl.

3. The compound of claim 1 wherein R₁ is ethyl.

4. The compound of claim 1 wherein R₁ is propyl.

5. The compound of claim 1 wherein R₁ is chloro.

6. The compound of claim 1 wherein R₂ is H.

7. The compound of claim 1 wherein R₃ is H.

8. A pharmaceutical composition comprising a compound of the formula: 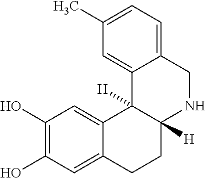
and a pharmaceutically acceptable carrier, wherein the compound is present in an amount sufficient to provide a dosage of about 0.5 to about 5 mg/kg.

9. The compound of claim 1, wherein the compound includes a stereocenter, and the compound is enantiomerically enriched in the stereoisomer correlating to the 6aR, 12bS absolute configuration.

10. The composition of claim 8, wherein the compound includes a stereocenter, and the compound is enantiomerically enriched in the stereoisomer correlating to the 6aR, 12bS absolute configuration.

11. A method for treating a dopamine-related dysfunction of the central nervous system evidenced by an apparent neurological, psychiatric, psychological, physiological, or behavioral disorder, said method comprising the step of administering a compound of claim 1 in an amount effective to reduce the symptoms of said disorder.

12. The method of claim 11, wherein the disorder is selected from the group consisting of Parkinson's disease, parkinsonism, restless leg syndrome, schizophrenia, Alzheimer's disease and other disorders with cognitive deficits (including age associated cognitive deficits,), substance abuse, autism, Huntington's chorea, tardive dyskinesia, attention deficit hyperactivity disorder and related developmental disorders, mood and anxiety disorders, Tourette's syndrome, neurodegeneration resulting from acute events like stroke, and renal or pulmonary dysfunction.

13. A method for treating a dopamine-related dysfunction of the central nervous system evidenced by an apparent neurological, psychiatric, psychological, physiological, or behavioral disorder, said method comprising the step of administering a composition of claim 8 in an amount effective to reduce the symptoms of said disorder.

14. The method of claim 13, wherein the disorder is selected from the group consisting of Parkinson's disease, parkinsonism, restless leg syndrome, schizophrenia, Alzheimer's disease and other disorders with cognitive deficits (including age associated cognitive deficits), substance abuse, autism, Huntington's chorea, tardive dyskinesia, attention deficit hyperactivity disorder and related developmental disorders, mood and anxiety disorders, Tourette's syndrome, neurodegeneration resulting from acute events like stroke, and renal or pulmonary dysfunction.

15. A pharmaceutical composition for treating dopamine-related dysfunction of the central nervous systems characterized by an apparent neurological, psychiatric, physiological, psychological, or behavioral disorder, said composition consisting essentially of a therapeutically effective amount of the compound according to claim 1 and a pharmaceutically-acceptable carrier.

16. A pharmaceutical composition for treating dopamine-related dysfunction of the central nervous systems characterized by an apparent neurological, psychiatric, physiological, psychological, or behavioral disorder, said composition consisting essentially of a therapeutically effective amount of the compound according to claim 8 and a pharmaceutically-acceptable carrier, wherein the compound is present in an amount sufficient to provide a dosage of about 0.5 to about 5 mg/kg.

17. A method of treating a dopamine-related dysfunction of the central nervous system characterized by an apparent neurological, psychiatric, physiological, psychological, or behavioral disorder in a patient suffering said CNS dysfunction, said method comprising the step of administering in an amount effective to reduce the symptoms of said disorder a compound according to claim 1 that has a duration of action sufficient for administration no more than three time daily and that causes typical or functionally selective activation of one of more dopamine receptors.

18. The method of claim 17, wherein the disorder is selected from the group consisting of Parkinson's disease, parkinsonism, restless leg syndrome, schizophrenia, Alzheimer's disease and other disorders with cognitive deficits (including age associated cognitive deficits,), substance abuse, autism, Huntington's chorea, tardive dyskinesia, attention deficit hyperactivity disorder and related developmental disorders, mood and anxiety disorders, Tourette's syndrome, neurodegeneration resulting from acute events like stroke, and renal or pulmonary dysfunction.

19. A method of treating a dopamine-related dysfunction of the central nervous system characterized by an apparent neurological, psychiatric, physiological, psychological, or behavioral disorder in a patient suffering said CNS dysfunction, said method comprising the step of administering in an amount effective to reduce the symptoms of said disorder a composition according to claim 8 that has a duration of action sufficient for administration no more than three-times daily and that causes typical or functionally selective activation of one of more dopamine receptors.

20. The method of claim 19, wherein the disorder is selected from the group consisting of Parkinson's disease, parkinsonism, restless leg syndrome, schizophrenia, Alzheimer's disease and other disorders with cognitive deficits (including age associated cognitive deficits), substance abuse, autism, Huntington's chorea, tardive dyskinesia, attention deficit hyperactivity disorder and related developmental disorders, mood and anxiety disorders, Tourette's syndrome, neurodegeneration resulting from acute events like stroke, and renal or pulmonary dysfunction.

21. A method of causing an improvement in cognitive function in individuals with no specific clinical deficit, said method comprising the step of administering in an amount effective to cause an improvement in cognitive function a compound according to claim 1.

22. A method of causing an improvement in cognitive function in individuals with no specific clinical deficit, said method comprising the step of administering in an amount effective to cause an improvement in cognitive function a composition according to claim 8.

23-32. (canceled)

33. The composition of claim 16, in the form of a transdermal formulation.

34. The composition of claim 16, in the form of an intranasal formulation.

35. The composition of claim 16, in the form of an oral disintegrating tablet.
